(19) United States
(12) Patent Application Publication
(10) Pub. No.: US 2020/0383660 A1
(43) Pub. Date: Dec. 10, 2020

(54) METHODS AND APPARATUSES FOR ULTRASOUND COUPLING

(71) Applicant: Butterfly Network, Inc., Guilford, CT (US)

(72) Inventors: Jonathan M. Rothberg, Guilford, CT (US); Tyler S. Ralston, Clinton, CT (US); Christopher Thomas McNulty, Guilford, CT (US); Nevada J. Sanchez, Guilford, CT (US)

(73) Assignee: Butterfly Network, Inc., Guilford, CT (US)

(21) Appl. No.: 16/908,905

(22) Filed: Jun. 23, 2020

Related U.S. Application Data
(63) Continuation of application No. PCT/US2019/015155, filed on Jan. 25, 2019.
(60) Provisional application No. 62/622,781, filed on Jan. 26, 2018.

(51) Int. Cl. A61B 8/00

(52) U.S. Cl. CPC A61B 8/4281; A61B 8/4411; A61B 8/4236

(57) ABSTRACT

Described herein are methods and apparatuses for ultrasound coupling. Certain aspects relate to coupling bodies for acoustically coupling an ultrasound device to a subject. A coupling body may include a first surface configured to couple to an ultrasound device, a second surface configured to contact the subject, a reservoir internal to the coupling body, and a plurality of openings extending between the reservoir and one or both of the first surface and the second surface. The reservoir may contain ultrasound gel. A coupling body may include an adhesive coupled to a subpart of the surface of the coupling body. A coupling body may include a first surface configured to contact the ultrasound device and a second surface including first adhesive configured to adhere to the subject. The first surface may also include second adhesive configured to adhere to an ultrasound patch device. Certain aspects also relate to packaging coupling bodies.

### CROSS-REFERENCE TO RELATED APPLICATIONS

The present application is a Continuation claiming the benefit of Patent Application Serial No. PCT/US2019/015155, filed Jan. 25, 2019, under Attorney Docket No. B1348.70071WO00, entitled “METHODS AND APPARATUSES FOR ULTRASOUND COUPLING,” which is hereby incorporated herein by reference in its entirety.

Patent Application Serial No. PCT/US2019/015155 claims the benefit under 35 U.S.C. § 119(e) of U.S. Provisional Patent Application Ser. No. 62/622,781, filed Jan. 26, 2018 under Attorney Docket No. B1348.70071US00 and entitled “METHODS AND APPARATUSES FOR ULTRASOUND COUPLING,” which is hereby incorporated herein by reference in its entirety.

### FIELD

Generally, the aspects of the technology described herein relate to ultrasound systems. Some aspects relate to methods and apparatuses for ultrasound coupling.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image.

Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a coupling body for acoustically coupling an ultrasound device to a subject includes a first surface configured to couple to the ultrasound device, a second surface configured to contact the subject, a reservoir internal to the coupling body, and a plurality of openings extending between the reservoir and one or both of the first surface and the second surface.

In some embodiments, the reservoir contains ultrasound gel. In some embodiments, the coupling body is configured to deposit the ultrasound gel through the plurality of openings from the reservoir to one or both of the first surface and the second surface in response to force applied to the body. In some embodiments, the ultrasound gel includes one or more humectants (e.g., 2 humectants, 3 humectants, or more). In some embodiments, one or more of the plurality of openings (and in some cases each opening) has a diameter between approximately 100 microns and 500 microns, including any value within that range. In some embodiments, the plurality of openings have a pitch (distance between openings) of approximately 1 mm-5 mm, including any value within that range.

In some embodiments, the coupling body is configured to mechanically maintain a coupling to the ultrasound device using at least one elastic, hook, latch, or screw. In some embodiments, the coupling body includes polypropylene. In some embodiments, the first surface and the second surface of the coupling body include a same material. In some embodiments, the coupling body includes an inner portion and an outer portion surrounding the inner portion, the first surface and the second surface are on the outer portion of the coupling body, and the reservoir is disposed between the inner portion and the outer portion. In some embodiments, the inner portion includes a hydrogel material. In some embodiments, the outer portion includes polypropylene.

In some embodiments, the coupling body further includes an adhesive coupled to a subpart of the first surface of the coupling body. In some embodiments, an area of the subpart of the first surface is substantially less than an area of the first surface. In some embodiments, the subpart includes a perimeter portion of the first surface of the coupling body. In some embodiments, the coupling body is substantially rectangular; the coupling body includes a middle portion, a first end portion, and a second end portion; and the adhesive is coupled to at least one of the first end portion and the second end portion and not the middle portion. In some embodiments, the middle portion, the first end portion, and the second end portion have substantially a same length. In some embodiments, the adhesive is configured to couple to a portion of the ultrasound device that does not include an acoustic lens of the ultrasound device.

In some embodiments, the coupling body further includes a middle portion, a first end portion, a second end portion, a first flexible member coupled between the middle portion and the first end portion, and a second flexible member coupled between the middle portion and the second end portion. In some embodiments, the first flexible member and the second flexible member include a flexible material having an elastic modulus between approximately 0.15 GPa and 14.7 GPa, including any value within that range. In some embodiments, the first flexible member and the second flexible member include hinges. In some embodiments, the first flexible member and the second flexible member each include at least one of the plurality of openings. In some embodiments, the adhesive is coupled to at least one of the first end portion and the second end portion and not the middle portion.

According to another aspect, a coupling body for acoustically coupling an ultrasound device to a subject includes a surface configured to couple to the ultrasound device and an adhesive coupled to a subpart of the surface of the coupling body. In some embodiments, an area of the subpart of the surface of the coupling body is substantially less than an area of the surface. In some embodiments, the subpart includes a perimeter portion of the surface of the coupling body. In some embodiments, the coupling body is substantially rectangular; the coupling body includes a middle portion, a first end portion, and a second end portion; and the adhesive is coupled to at least one of the first end portion and the second end portion and not the middle portion. In some embodiments, the middle portion, the first end portion, and the second end portion have substantially a same length.

In some embodiments, the coupling body is substantially rectangular; and the coupling body includes a middle portion, a first end portion, a second end portion, a first flexible member coupled between the middle portion and the first end portion, and a second flexible member coupled between the middle portion and the second end portion. In some embodiments, the first flexible member and the second flexible member include a flexible material having an elastic modulus between approximately 0.15 GPa and 14.7 GPa, including any value within that range. In some embodiments, the first flexible member and the second flexible member include hinges. In some embodiments, the adhesive is coupled to at least one of the first end portion and the second end portion and not the middle portion. In some embodiments, the adhesive is configured to couple to a portion of the ultrasound device that does not include an acoustic lens of the ultrasound device.

According to another aspect, a coupling body for acoustically coupling an ultrasound device to a subject includes a middle portion, a first end portion, a second end portion, a first flexible member coupled between the middle portion and the first end portion, and a second flexible member coupled between the middle portion and the second end portion. In some embodiments, the first flexible member and the second flexible member include a flexible material having an elastic modulus between approximately 0.15 GPa and 14.7 GPa, including any value within that range. In some embodiments, the first flexible member and the second flexible member include hinges.

According to another aspect, a coupling body for acoustically coupling an ultrasound device to a subject includes a first surface configured to contact the ultrasound device and a second surface including first adhesive configured to adhere to the subject. In some embodiments, the first surface includes second adhesive configured to adhere to the ultrasound device, which is an ultrasound patch device.

In some embodiments, the coupling body further includes a reservoir internal to the coupling body and a plurality of openings extending between the reservoir and one or both of the first surface and the second surface. In some embodiments, the reservoir contains ultrasound gel. In some embodiments, the coupling body is configured to deposit the ultrasound gel through the plurality of openings from the reservoir to one or both of the first surface and the second surface in response to force applied to the body. In some embodiments, the ultrasound gel includes one or more humectants (e.g., 2 humectants, 3 humectants, or more). In some embodiments, each of the plurality of openings is approximately 100-500 microns in diameter. In some embodiments, the plurality of openings have a pitch of between approximately 1 mm and 5 mm, including any value within that range.

In some embodiments, the coupling body includes polypropylene. In some embodiments, the first surface and the second surface of the coupling body include a same material. In some embodiments, the coupling body includes an inner portion and an outer portion surrounding the inner portion, the first surface and the second surface are on the outer portion of the coupling body, and the reservoir is disposed between the inner portion and the outer portion. In some embodiments, the inner portion includes a hydrogel material. In some embodiments, the outer portion includes polypropylene.

In some embodiments, the second adhesive is coupled to a subpart of the first surface of the coupling body. In some embodiments, an area of the subpart of the first surface of the coupling body is substantially less than an area of the first surface. In some embodiments, the subpart includes a perimeter portion of the first surface of the coupling body.

According to another aspect, a method of packaging an ultrasound coupling body includes: providing a coupling body including a reservoir internal to the coupling body, a first surface, and a second surface; adhering a first seal over the first surface; adhering a second seal over the second surface; and sealing the coupling body in a package.

In some embodiments, the first surface includes a first adhesive and a first plurality of openings extending between the reservoir and the first surface, the second surface includes a second plurality of openings extending between the reservoir and the second surface, adhering the first seal over the first surface includes adhering the first seal over the first plurality of openings and the adhesive on the first surface, and/or adhering the second seal over the second surface includes adhering the second seal over the second plurality of openings on the second surface. In some embodiments, the second surface includes a second adhesive, and adhering the second seal includes adhering the second seal over the second plurality of openings and the second adhesive on the second surface. In some embodiments, the first surface is configured to adhere to an ultrasound patch device.

In some embodiments, the reservoir contains ultrasound gel. In some embodiments, the ultrasound gel includes one or more humectants (e.g., 2 humectants, 3 humectants, or more). In some embodiments, each of the first plurality of openings and the second plurality of openings has a diameter between approximately 100 microns and 500 microns, including any value in that range. In some embodiments, the first plurality of openings and the second plurality of openings have a pitch of between approximately 1 mm and 5 mm, including any value within that range. In some embodiments, the coupling body includes polypropylene. In some embodiments, the first surface and the second surface of the coupling body include a same material. In some embodiments, the coupling body includes an inner portion and an outer portion surrounding the inner portion; the first surface and the second surface are on the outer portion of the coupling body; and the reservoir is disposed between the inner portion and the outer portion. In some embodiments, the inner portion includes a hydrogel material. In some embodiments, the outer portion includes polypropylene. In some embodiments, the adhesive is coupled to a subpart of the first surface of the coupling body. In some embodiments, an area of the subpart of the first surface is substantially less than an area of the first surface. In some embodiments, the subpart includes a perimeter portion of the first surface of the coupling body. In some embodiments, the coupling body is substantially rectangular; the coupling body includes a middle portion, a first end portion, and a second end portion; and the adhesive is coupled to at least one of the first end portion and the second end portion and not the middle portion. In some embodiments, the middle portion, the first end portion, and the second end portion have substantially a same length.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows an example coupling body configured to adhere to an ultrasound device in accordance with certain embodiments described herein;

FIG. 2 shows an example view of the subject-facing surface of the coupling body of FIG. 1 when packaged in accordance with certain embodiments described herein;

FIG. 3 shows an example view of the device-facing surface of the coupling body of FIG. 1 when packaged in accordance with certain embodiments described herein;

FIG. 4 shows an example view of the subject-facing surface of a coupling body configured to adhere to a subject in accordance with certain embodiments described herein;

FIG. 5 shows an example view of the device-facing surface of the coupling body of FIG. 4 in accordance with certain embodiments described herein;

FIG. 6 shows an example view of the device-facing surface of the coupling body of FIG. 4 when packaged in accordance with certain embodiments described herein;

FIG. 7 shows an example view of the subject-facing surface of the coupling body of FIG. 4 when packaged in accordance with certain embodiments described herein;

FIG. 8 shows an example of an ultrasound patch in accordance with certain embodiments described herein;

FIG. 9 shows an example of the patch of FIG. 8 coupled to a patient in accordance with certain embodiments described herein;

FIG. 10 shows an example exploded view of the patch of FIG. 8 in accordance with certain embodiments described herein;

FIG. 11 shows an example “bottom up” exploded view of the patch of FIG. 8 in accordance with certain embodiments described herein;

FIG. 12 shows an example cross-section of a coupling body in accordance with certain embodiments described herein;

FIG. 13 shows an example cross-section of a coupling body in accordance with certain embodiments described herein;

FIG. 14 shows an example cross-section of a coupling body in accordance with certain embodiments described herein;

FIG. 15 shows an example cross-section of a coupling body in accordance with certain embodiments described herein;

FIG. 16 shows an example cross-section of a coupling body 1602 in accordance with certain embodiments described herein;

FIG. 17 shows an example cross-section of a coupling body 1702 in accordance with certain embodiments described herein;

FIG. 18 shows an example cross-section of a coupling body 1802 in accordance with certain embodiments described herein;

FIG. 19 shows an example cross-section of a coupling body 1902 in accordance with certain embodiments described herein; and

FIG. 20 shows an example process for packaging a coupling body.

### DETAILED DESCRIPTION

Ultrasound gel is typically applied to an acoustic lens area of an ultrasound imaging device prior to imaging a subject with the ultrasound imaging device. The ultrasound gel may help to reduce air gaps between the ultrasound imaging device and the subject and thereby establish acceptable impedance matching coupling for ultrasound signal transmission and reception. However, ultrasound gel may be messy and uncomfortable for the subject, require cleaning of the ultrasound imaging device and the subject after every use, and need to be replenished during an imaging session.

The inventors have recognized that a coupling body may be used instead of ultrasound gel. In particular, one surface of the coupling body may be configured to adhere to an ultrasound imaging device and another surface of the coupling body may be configured to contact a subject. Accordingly, the coupling body may interpose between the ultrasound imaging device and the subject during imaging. The coupling body may be formed of any acoustically transparent material such that, when the coupling body interposes between the ultrasound imaging device and the subject, the coupling body establishes acceptable impedance matching coupling for ultrasound signal transmission and reception. In some embodiments, the coupling body includes a material having a characteristic acoustic impedance differing from that of water by at most 6×10⁵ N·s/m³ (e.g., by at most 5×10⁵ N·s/m³). The characteristic acoustic impedance of water is approximately 1.494×10⁶ N·s/m³, and accordingly the coupling body may include as non-limiting examples polypropylene, polydimethylsiloxane (PDMS), and/or agar (which have characteristic acoustic impedances of approximately 2.002×10⁶ N·s/m³, 1.1×10⁶ N·s/m³, and 1.52-1.76×10⁶ N·s/m³, respectively). In some embodiments, the coupling body may include a hydrogel (e.g., 2-hydroxyethyl methacrylate), room-temperature-vulcanizing silicone, a Sylgard® silicone, or polystyrene.

The inventors have also recognized that one surface of a coupling body may be configured to adhere to a subject and another surface of the coupling body may be configured to contact an ultrasound imaging device. Adhering the coupling body to a subject may be useful, for example, when ultrasound imaging of a specific anatomical feature must be performed over an extended period of time. A user may search, with an ultrasound imaging device, for a location on the subject where the anatomical feature can be imaged, and once the location is found, the user may adhere the coupling body to that location. Henceforth, the coupling body may serve as a landmark for where the ultrasound imaging device should be placed to image the anatomical feature of interest.

The inventors have also recognized that one surface of a coupling body may be configured to adhere to a subject and another surface of the coupling body may be configured to adhere to an ultrasound patch device. Once the ultrasound path device is adhered to the coupling body on the subject, the ultrasound patch may remain in place on the subject, capable of imaging continuously and/or periodically. A user may press the ultrasound patch device that is adhered to the subject against the subject in order to improve acoustic coupling between the ultrasound patch device and the subject when the ultrasound patch device is collecting ultrasound data.

The inventors have also recognized that it may be helpful for a coupling body to include an internal reservoir configured to contain ultrasound gel, as well as openings in the surfaces of the coupling body configured to contact/adhere to the ultrasound imaging device and the subject. Accordingly, ultrasound gel may flow from the internal reservoir, out of the coupling body through the openings, and onto the ultrasound imaging device and the subject. The ultrasound gel may be an acoustically transparent material and may fill air gaps between the coupling body and the subject and/or the ultrasound imaging device during imaging, thereby improving impedance matching coupling for ultrasound signal transmission and reception between the subject and the ultrasound imaging device. The openings may be configured to release ultrasound gel from the one or more internal reservoirs in response to a user pressing down on the ultrasound imaging device. The diameter of the openings may be selected to be small enough such that the user can control, based on force applied, how much ultrasound gel the openings release with acceptable precision. Furthermore, the coupling body may be configured to allow the user to selectively apply ultrasound gel in certain locations, for example by pushing down with the ultrasound imaging device on certain portions of the coupling body but not others. This may help the user to apply a sufficient amount of ultrasound gel to obtain an image of acceptable quality, without applying too much ultrasound gel, which can be messy and uncomfortable. The coupling body may be configured to not adhere to the lens of the ultrasound imaging device, such that ultrasound gel from the internal reservoir may flow to the lens and create a lubricious interface. For example, there may only be adhesive on a subpart of the surface of the coupling body configured to adhere to the ultrasound imaging device. This lubricious interface may help a user of the ultrasound imaging device to expel air bubbles at the interface when the user presses down on the ultrasound imaging device.

The inventors have also recognized that it may be helpful for the coupling body to include flexible members between various portions of the coupling body such that various portions of the coupling body may rotate independently of other portions. This may be helpful for ensuring tight coupling of the coupling body to the ultrasound imaging device and for enabling use of the coupling body with a variety of ultrasound imaging devices having different shapes (e.g., linear, phased array, curvilinear, transesophageal, and transvaginal ultrasound imaging devices).

The inventors have also recognized that a coupling body may be packaged with seals covering openings and adhesives on the surfaces of the coupling body. The seals may therefore be configured to prevent ultrasound gel from exiting the coupling body through the openings before use, and to prevent the adhesive from being damaged (e.g., adhering to other items, losing stickiness, etc.) prior to use.

Conventional ultrasound devices are limited because each of them operates at just a single one of several medically-relevant frequency ranges. For example, some conventional ultrasound devices operate only at frequencies in the range of 1-3 MHz (e.g., for applications such as obstetric, abdomen and gynecological imaging), whereas other conventional devices operate only at frequencies in the range of 3-7 MHz (e.g., for applications such as breast, vascular, thyroid, and pelvic imaging). Still other conventional ultrasound probes operate only at frequencies in the range of 7-15 MHz (e.g., for applications such as musculoskeletal and superficial vein and mass imaging). Since higher frequency ultrasound signals attenuate faster in tissue than lower frequency ultrasound signals, conventional devices operating only at higher frequencies are used for generating images of a patient at shallow depths (e.g., 5 cm or less) for applications such as central line placement or the aforementioned imaging of superficial masses located just beneath the skin. On the other hand, conventional devices operating only at lower frequencies are used to generate images of a patient at greater depths (e.g., 10-25 cm) for applications such as cardiac and kidney imaging. As a result, a medical professional may need to use multiple different probes, which is inconvenient and expensive, as it requires procuring multiple different probes configured to operate at different frequency ranges.

Recently, universal ultrasound devices have been introduced. In particular, such devices are configured to operate at multiple different medically-relevant frequency ranges and image patients at a sufficiently high resolution for forming medically-relevant images at a wide range of depths. Accordingly, multiple conventional ultrasound probes may all be able to be replaced by a single universal ultrasound device, and medical professionals or other users may use a single universal ultrasound probe to perform multiple imaging tasks instead of using a multitude of conventional ultrasound probes each having limited applicability. Such imaging devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), which is incorporated by reference herein in its entirety.

The inventors have recognized that coupling bodies that are configured to adhere to an ultrasound device in accordance with certain embodiments described therein may be especially appropriate for use with such universal ultrasound devices. A coupling body may adhere to a universal ultrasound device and help to establish suitable acoustical coupling for the duration of an ultrasound imaging session, which may include imaging of multiple anatomical areas.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

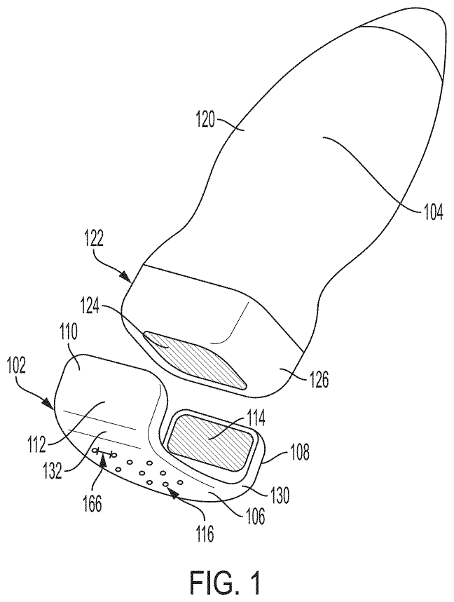

FIG. 1 shows an example coupling body 102 configured to adhere to an ultrasound device 104 in accordance with certain embodiments described herein. The coupling body 102 includes a middle portion 106, a first end portion 108, a second end portion 110, a first flexible member 130, and a second flexible member 132. The coupling body 102 further has a subject-facing surface 112 and a device-facing surface 114. The subject-facing surface 112 is on an opposite side of the coupling body 102 as the device-facing surface 114. The first flexible member 130 is coupled between the middle portion 106 and the first end portion 108. The second flexible member 132 is coupled between the middle portion 106 and the second end portion 110. The middle portion 106 of the coupling body 102 includes openings 116 on the subject-facing surface 112 and openings on the device-facing surface 114 (not visible in FIG. 1). The device-facing surface 114 of the first end portion 108 and the second end portion 110 includes adhesive (not visible in FIG. 1). The ultrasound device 104 includes a body portion 120 and a head portion 122. The head portion 122 includes an acoustic lens 124 and a lens housing 126.

The device-facing surface 114 of the coupling body 102 is configured to couple to the ultrasound device 104. In particular, the adhesive on the device-facing surface 114 of the first end portion 108 and the second end portion 110 of the coupling body 102 is configured to adhere to the lens housing 126 or the body portion 120 of the ultrasound device 104. When the coupling body 102 is coupled to the ultrasound device 104, the device-facing surface 114 of the middle portion 106 of the coupling body 102 faces the acoustic lens 124. The subject-facing surface 112 of the coupling body 102 is configured to contact a subject during ultrasound imaging with the ultrasound device 104. Accordingly, the coupling body 102 is configured to interpose between the ultrasound device 104 and the subject during imaging.

The coupling body 102 may be formed of any acoustically transparent material such that, when the coupling body 102 interposes between the ultrasound device 104 and the subject, the coupling body 102 establishes acceptable impedance matching coupling for ultrasound signal transmission and reception. In some embodiments, the coupling body 102 includes a material having a characteristic acoustic impedance differing from that of water by at most 6×10⁵ N·s/m³ (e.g., by at most 5×10⁵ N·s/m³). The characteristic acoustic impedance of water is approximately 1.494×10⁶ N·s/m³, and accordingly the coupling body 102 may include as non-limiting examples polypropylene, polydimethylsiloxane (PDMS), and/or agar (which have characteristic acoustic impedances of approximately 2.002×10⁶ N·s/m³, 1.1×10⁶ N·s/m³, and 1.52-1.76×10⁶ N·s/m³, respectively). In embodiments in which the coupling body 102 includes PDMS and/or agar, to improve the mechanical durability of the coupling body 102, the PDMS and/or agar may be cured around a mechanically reinforcing screen or mesh. The screen or mesh may be made of, for example, nylon, polyester, polyvinylidene fluoride, polypropylene, ultrahigh molecular weight polyethylene, polytetrafluoroethylene, stainless steel, silk, or any other biologically inert/biocompatible materials, and may include large openings to allow an acceptable level of ultrasound penetration and thin diameter filaments for reinforcement.

In some embodiments, the coupling body 102 may include a hydrogel. For example, the hydrogel may be formed of a hydrophilic monomer/polymer, crosslinking agent, and catalyst. The hydrophilic monomer/polymer may include, for example, methacrylic acid, salts of methacrylic acid, esters of methacrylic acid, salts and acids of esters of methacrylic acid, amides of methacrylic acid, N-alkyl amides of methacrylic acid, salts and acids of N-alkyl amides of methacrylic acid, N-vinylpyrrolidone, acrylamide, acrylamide derivatives, methacrylamide, methacrylamide derivatives, acrylamide, N-isopropylacrylamide, 2-hydroxyethyl methacrylate (HEMA), 2-hydroxypropyl methacrylate, acrylic acid, 2-acrylamido-2-methyl-1-propanesulfonic acid, 3-sulfopropyl acrylate potassium salt, 2-(acryloyloxy)ethyl]trimethylammonium methyl sulfate and its inorganic salts, 2-(methacryloyloxy)ethyl]trimethylammonium methyl sulfate and its inorganic salts, or any combination thereof. The crosslinking agent may include, for example, ethylene glycol dimethacrylate, ethylene glycol diacrylate, poly(ethylene glycol)dimethacrylate, poly(ethylene glycol)diacrylate, poly(ethylene glycol)diacrylamide, N,N′-methylenebisacrylamide, piperazine diacrylamide, glutaraldehyde, epichlorohydrin, crosslinking agents containing 1,2-diol structures, crosslinking agents containing functionalized peptides, and crosslinking agents containing proteins. The catalyst may include, for example, benzoyl peroxide, ammonium persulfate, sodium bisulfite, potassium persulfate, sodium persulfate, and the potassium salt of persulfate. In some embodiments, the coupling body 102 may further include a water-soluble polymer, such as polyvinylpyrrolidone, polyethylene glycol, or polyethylene oxide, in addition to a hydrogel. In some embodiments, the coupling body 102 may include room-temperature-vulcanizing (RTV) silicone (e.g., RTV 6020), a Sylgard® silicone, or polystyrene.

The subject-facing surface 112 and the device-facing surface 114 may include the same material and have similar properties, which may improve the ease of manufacturing the coupling body 102.

The coupling body 102 contains ultrasound gel in one or more internal reservoirs. The openings 116 on the subject-facing surface 112 of the middle portion 106 extend from the subject-facing surface 112 to the one or more internal reservoirs. Similarly, the openings on the device-facing surface 114 of the middle portion 106 extend from device-facing surface 114 to the one or more internal reservoirs. Accordingly, ultrasound gel may flow from the one or more internal reservoirs, out of the coupling body, and onto the acoustic lens 124 (through the openings in the device-facing surface 114) and the subject (through the openings 116 in the subject-facing surface). The ultrasound gel may be an acoustically transparent material and may fill air gaps between the coupling body 102 and the subject and/or the ultrasound device 104 during imaging, thereby improving impedance matching coupling for ultrasound signal transmission and reception between the subject and the ultrasound device 104. In some embodiments, the ultrasound gel may include, for example, any combination of propylene glycol, glycerine, phenoxyethanol, and oils. In some embodiments, the ultrasound gel may be a commercially available ultrasound gel, such as Aquasonic® ultrasound gels.

The coupling body 102 may be configured to deposit the ultrasound gel through the openings 116 from the one or more internal reservoirs to the subject-facing surface 112 and the device-facing surface 114 in response to a user pressing down on the ultrasound device 104 when the ultrasound device 104 is in contact with a subject (and, as a consequence pressing down on the coupling body 102). There are a number of considerations for choosing the diameter of the openings 116:

1. The diameter of the openings 116 may be selected to be small enough such that the user can control, based on force applied, how much ultrasound gel the openings 116 release with acceptable precision. For example, when a user presses down on the coupling body 102 with a typical amount of force, the flow rate of ultrasound gel from the openings 116 should not be unacceptably high. In some embodiments, the flow rate of ultrasound gel from the openings 116 may be between or equal to 0.1 mL/sec and 10 mL/sec (e.g., 0.1 mL/sec, 1 mL/sec, 10 mL/sec).

2. The diameter of the openings 116 may be selected to be large enough such that the amount of pressure required to eject a relatively small amount of ultrasound gel through the openings 116 is not unacceptably large, for example, large enough to cause the subject discomfort. In some embodiments, acceptable ranges of pressures may be between or equal to 1 kPa and 5 kPa, 1 kPa and 10 kPa, 1 kPA and 50 kPa, 1 kPa and 100 kPa, or 1 kPa and 500 kPa.

3. Selection of the diameter of the openings 116 may depend on the viscosity of the ultrasound gel, as viscosity of the ultrasound gel may affect the flow rate of ultrasound gel through an opening of given diameter in response to a given amount of force. In some embodiments, the viscosity of the ultrasound gel may be approximately 25,000-195,00 cps. In some embodiments, instead of a gel, a less viscous coupling medium (e.g., more akin to a liquid) may be used. Use of a less viscous coupling medium may be feasible when the coupling medium may be dispensed from a reservoir within the coupling body 102 with some degree of precision.

In some non-limiting embodiments, appropriate parameters for the diameter of the openings 116 may be estimated given the viscosity of the ultrasound gel, typical pressures applied to the coupling body 102, desired flow rate of ultrasound gel from the openings 116, and other structural dimensions of the coupling body 102 using Poiseuille's Equation for laminar flow of liquid through a pipe: ΔP=(8μLQ)/(πR⁴), where ΔP is the pressure difference between the two ends of the pipe, L is the length of pipe, μ is the dynamic viscosity of the liquid, Q is the volumetric flow rate of the liquid, and R is the pipe radius. That is, in some embodiments, at least one of the openings or each of the openings may be considered a pipe, although not all embodiments are limited in this respect. In the specific context of applying Poiseuille's equation to the openings 116, ΔP is the pressure of the ultrasound device 104 against the patient, L is the height of the openings 116 (i.e., along the dimension of the coupling body 102 corresponding to the thickness of the coupling body 102), μ is the dynamic viscosity of the ultrasound gel, Q is the rate at which the ultrasound gel is dispensed from the openings 116, and R is the radius of the openings 116 (i.e., half the diameter of the openings 116).

4. The diameter of the openings 116 may be selected to be small enough such that the ultrasound gel does not dehydrate at an unacceptable rate. For example, if the coupling body 102 is configured to be usable for 1 day, 2 days, or three days, the diameter of the openings 116 should be large enough such that the ultrasound gel is not rendered unusable due to dehydration when the ultrasound gel is exposed to the environment through the openings 116 for 1 day, 2 days, or 3 days. The diameter of the openings 116 may also be selected based on how long the coupling body 102 needs to remain usable. For example, a coupling body 102 configured to adhere to an ultrasound device 104 for a single imaging device may have a larger diameter for the openings 116 than a coupling body 102 configured to adhere to a patient and an ultrasound patch device (as discussed further herein) for a prolonged period of time (e.g., hours, days, or weeks).

In some embodiments, the above considerations may be satisfied by openings 116 that are, for example, approximately 1000 microns, 900 microns, 800 microns, 700 microns, 600 microns, 500 microns, 400 microns, 300 microns, 200 microns, or 100 microns in diameter, between 100 microns and 1000 microns, or any value within those ranges. In some embodiments, the openings have a diameter of 500 microns. In other embodiments, the above considerations may be satisfied by openings 116 having a diameter that is larger than 1000 microns, or smaller than 100 microns.

In FIG. 1, the openings 116 are positioned on portions of the subject-facing surface 112 of the coupling body 102 that face the acoustic lens 124, and may therefore be positioned along the path of acoustic waves transmitted through the acoustic lens. This may help the user to dispense ultrasound gel in the direct path of the acoustic waves, where acoustic coupling is needed. However, in other embodiments, the openings 116 may not be positioned on portions of the subject-facing surface 112 of the coupling body 102 that are along the path of acoustic waves transmitted through the acoustic lens, but may be positioned on portions of the subject-facing surface 112 of the coupling body 102 that are adjacent to but not facing the acoustic lens 124 (i.e., adjacent but outside of the periphery of the acoustic lens 124). This may help to reduce interference in propagation of the acoustic lens caused by the openings 116. In such embodiments, once ultrasound gel is dispensed through the openings 116 near the periphery of the acoustic lens 124, a user may use the ultrasound device 124 to spread the ultrasound gel into a desired position (e.g., between the subject and the acoustic lens 124).

The coupling body 102 may also be configured to allow the user to selectively apply ultrasound gel in certain locations, for example by pushing down with the ultrasound device 104 on certain portions of the coupling body 102 but not others. This may help the user to apply a sufficient amount of ultrasound gel to obtain an image of acceptable quality, without applying too much ultrasound gel, which can be messy and uncomfortable. The pitch of the openings 116 may be selected to be small enough (e.g. 5 mm, 4 mm, 3 mm, 2 mm, 1 mm, or any suitable pitch) such that there are a sufficient number of openings 116 in the coupling body 102 to release a sufficient amount of ultrasound gel for collecting images of acceptable quality. Additionally, range gating may help reduce reflections of acoustical waves from the openings 116 back to the ultrasound device 104. Such a diffraction effect may occur where the pitch of the openings 116 is less than about a wavelength of the acoustical waves, as the waves may then coherently interfere. By spacing the openings such that there is no less than a wavelength of kerf, it may be possible to avoid diffraction effects. Scattering and diffraction caused by the openings 116 may lead to an attenuated acoustics pressure wave, thus to minimize this transmission loss, the perforated material is selected to impedance match well with the ultrasound gel, the subject, and the acoustic lens 124. In some embodiments, the pitch 166 of the openings 116 may be less than 1 mm, or greater than 5 mm. The openings 116 may be formed in a material (e.g., a hydrogel or polypropylene) that is sufficiently rigid such that the openings 116 are configured to substantially resist deformation in response to force applied to the coupling body 102. In some embodiments, the openings 116 may be reinforced with a material (e.g., a metal) to provide this rigidity. In some embodiments, the thickness of the coupling body 102 may be selected to be thin enough to limit, to an acceptable degree, acoustic pressure losses and ultrasound artifacts due to the coupling body 102, while also maintaining acceptable rigidity under the maximal stresses and temperatures that an ultrasound device 104 may experience. In some embodiments, the coupling body may have a thickness between or equal to 50 microns and 1 cm (e.g., between or equal to 50 microns and 4 mm, between or equal to 500 microns and 6 mm, between or equal to 3 mm and 6 mm). In some embodiments, a thickness of 500 microns may be suitable for the thickness of the coupling body 102. Additionally, the material of the coupling body 102 may be modified to provide sufficient rigidity. In particular, the elastic modulus and shear strength of the coupling body 102 may be modulated by layering different materials in the coupling body 102 (e.g., layering RTV silicone and polypropylene) and modulating their thicknesses. Furthermore, the size and pitch of the openings 116 may affect the elastic modulus and shear strength of the coupling body 102. In embodiments in which the coupling body 102 includes PDMS and/or agar, mechanically rigidity may be improved by curing the PDMS and/or agar around a mechanically reinforcing screen or mesh. The screen or mesh may be made of, for example, nylon, polyester, polyvinylidene fluoride, polypropylene, ultrahigh molecular weight polyethylene, polytetrafluoroethylene, stainless steel, silk, or any other biologically inert/biocompatible materials, and include large openings to allow an acceptable level of ultrasound penetration and thin diameter filaments for reinforcement.

In some embodiments, similar features apply to the openings in the device-facing surface 114. As discussed above, the adhesive on the device-facing surface 114 is configured to adhere to the lens housing 126 but not to the acoustic lens 124. Because the middle portion 106 of the coupling body 102 includes the openings on the device-facing surface 114, ultrasound gel from the one or more internal reservoirs may flow to the acoustic lens 124 and create a lubricious interface. This lubricious interface may help a user of the ultrasound device 104 to expel air bubbles at the interface when the user presses down on the ultrasound device 104. The adhesive on the device-facing surface 114 may be, for example, a pressure-sensitive adhesive (PSA). The PSA may include, for example, acrylics, butyl rubbers, natural rubber, nitrile rubber, ethylene-vinyl acetate, silicone rubbers, and styrene-rubber block copolymers. In some embodiments, the pressure sensitive adhesive requires less pressure to adhere to the coupling device than a threshold pressure for ultrasound gel to flow through the openings in the coupling body. In some embodiments, the adhesive may be any pressure sensitive adhesive (e.g., under circumstances where the first end portion and the second end portion comprise the adhesive and the middle portion does not comprise the adhesive).

In some embodiments, the ultrasound gel may include lubricants, such as oils, soaps, surfactants, and emulsifiers. The lubricants may help to increase the lubricity of the subject-facing surface 112 of the coupling body 102, thereby helping the coupling body 102 to glide across the subject's skin during imaging and to be repositioned on the subject. In some embodiments, the ultrasound gel may include one or more humectants (hygroscopic substances used to retain moisture). A humectant may comprise one or more hydrophilic chemical groups (e.g., hydroxyl groups, amines, carboxyl groups, esters). The one or more hydrophilic groups in a humectant may be configured to form one or more hydrogen bonds with one or more molecules of water. Non-limiting examples of humectants include triethylene glycol, tripropylene glycol, propylene glycol, polypropylene glycols, glycerin, hexylene glycol, butylene glycol, urea, collagen, aloe vera gel, alpha hydroxyl acids (e.g., glycolic acid, lactic acid, malic acid, citric acid, tartaric acid), polyols (e.g., polydextrose, sugar alcohols (e.g., sorbitol, glycerol, xylitol, maltitol)), sodium hexametaphosphate, honey, glucose syrup, sucrose, egg yolk, egg white, glycerin triacetate, algae extract, hyaluronic acid, arnica extract, Baobab protein, caprylyl glycol, calendula extract, ceramides, colloidal oatmeal, glycoproteins, elastins, keratin, jojoba protein, hydrolyzed silk protein, ascorbates, tocopherols, hydrolyzed wheat protein, witch hazel extract, rhubarb root extract, components of soapbark (e.g., quillaia), salts (e.g., sodium chloride), cellulose fibers (e.g., cotton and paper), and wood. In some embodiments, the ultrasound gel may include preservatives, antiseptics, anti-fungals, anti-bacterials, bactericidal and bacteriostatic agents (e.g., alcohols, hydrogen peroxide, and organic or inorganic salts), and/or organic acids (e.g., citric acid, lactic acid, and acetic acid). Such substances may help to keep mold, fungus, and microbes out of the ultrasound gel by killing them or inhibiting/slowing/stalling their growth. This in turn may help to prevent infections, passing of diseases, spread of illnesses, etc. As will be discussed below, in some embodiments the coupling body 102 may not contain internal reservoirs for ultrasound gel, and may lack the openings 116.

The portions of the coupling body 102 having adhesive on them may be chosen for ease of use and sufficient adhesion. For example, too little adhesive may result in poor adhesion, while too much adhesive may be difficult or tedious for a user to adhere to the ultrasound device 104. In some embodiments, the adhesive may be on the entire device-facing surface 114 of the first end portion 108 and the second end portion 110. In some embodiments, the adhesive may be on a subpart (e.g., in the range of 5%-95%) of the device-facing surface 114 of the coupling body 102. The subpart of the device-facing surface 114 on which the adhesive is disposed may have an area that is substantially less than the area of the entire device-facing surface 114. For example, the adhesive may be only on the device-facing surface 114 of the first end portion 108 and the second end portion 110 (e.g., approximately 55-70% of the device-facing surface 114 of the coupling body 102) or on a perimeter portion of the device-facing surface 114 of the first end portion 108 and the second end portion 110 (e.g., approximately 5-10% of the device-facing surface 114 of the coupling body 102). While adhesive may not be on the device-facing surface 114 of the middle portion 106 in some embodiments, in other embodiments, the adhesive may also be on a perimeter portion of the device-facing surface 114 of the middle portion 116 (e.g., approximately 75-85% of the device-facing surface 114 of the coupling body 102), where the adhesive may adhere to the lens housing 126 rather than the acoustic lens 124. In still other embodiments, the adhesive may be on the entire device-facing surface 114 of the middle portion 106. The adhesive may include pastes, stick or semi-sticky gels, non-water soluble polymers, hydrophobic substances, and/or tacky substances. The adhesive may be reversible, in other words, the coupling body 102 may be removed from the ultrasound device 104 after having been adhered to the ultrasound device 104.

In some embodiments, other methods for coupling the coupling body 102 to the ultrasound device 104 may be used. For example, the coupling body 102 may include an elastic band at its perimeter that is configured to secure around the ultrasound device 104 (either at the body portion 120 or the head portion 122). As another example, a separate elastic band may be wrapped around the coupling body 102 and the ultrasound device 104 (either at the body portion 120 or the head portion 122) to secure the coupling body 102 to the ultrasound device 104. In some embodiments, the coupling body 102 may include hooks configured to hook onto loops on the ultrasound device 104. In other embodiments, the coupling body 102 may include loops configured to loop around hooks on the ultrasound device 104. As further examples, the coupling body 102 and the ultrasound device 104 may include a latch for securing the coupling body 102 to the ultrasound device 104, or the coupling body 102 and the ultrasound device 104 may include screws and screw holes for securing the coupling body 102 to the ultrasound device 104.

The first flexible member 130 is coupled between the middle portion 106 of the coupling body 102 and the first end portion 108 of the coupling body 102. The second flexible member 132 is coupled between the middle portion 106 of the coupling body 102 and the second end portion 110 of the coupling body 102. The first flexible member 130 and the second flexible member 132 may be configured as hinges. In particular, the first flexible member 130 may be configured to enable the first end portion 108 to rotate about the first flexible member 130 substantially independently of the middle portion 106. Similarly, the second flexible member may be configured to enable the second end portion 110 to rotate about the second flexible member 132 substantially independently of the middle portion 106. Therefore, the first end portion 108 and the second end portion 110 may rotate with respect to the middle portion 106 to assume a shape that conforms to the head portion 122 of the ultrasound device 104. This may be helpful for ensuring tight coupling of the coupling body 102 to the ultrasound device 104 and for enabling use of the coupling body 102 with a variety of ultrasound devices having different shapes (e.g., linear, phased array, curvilinear, transesophageal, and transvaginal ultrasound devices). The first flexible member 130 and the second flexible member 132 may include any type of flexible material, such as an elastic material, a polymer, polystyrene, polypropylene, or aluminum foil. In some embodiments, the elastic modulus of the flexible material may be in the range of approximately 0.15 GPa and 14.7 GPa. In some embodiments, the shear modulus of elasticity of the flexible material may be between or equal to 10 Pa and 30 GPa (e.g., 1000 Pa, 250 kPa, 26 GPa). In some embodiments, the flexible material is made from the same material as a material of the first end portion, the second end portion, and/or the middle portion. In some embodiments, the primary material of the coupling body 102 itself may allow the coupling body 102 to assume a shape that conforms to the ultrasound device 104, and the first flexible member 130 and the second flexible member 132 may not be necessary.

While the coupling body 102 is shown as substantially rectangular, other shapes may be possible. For example, the shape of the coupling body 102 may be configured to conform to a specific ultrasound device 104. While the middle portion 106, the first end portion 108, and the second end portion 110 are shown as being of substantially the same length, in some embodiments the middle portion 106, the first end portion 108, and/or the second end portion 110 may have different lengths. Furthermore, the coupling body 102 may take on a variety of sizes, which in some embodiments may depend on the size of a specific ultrasound device 104.

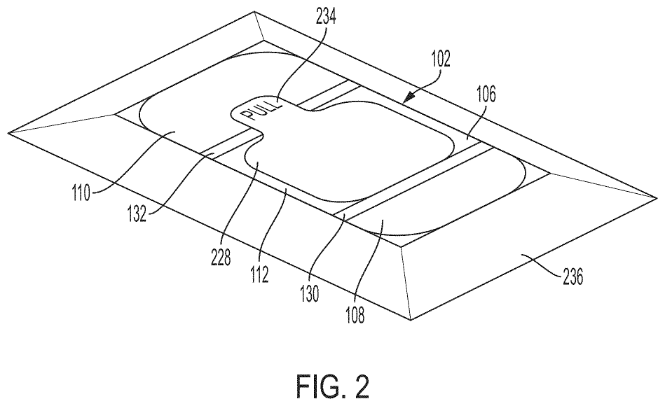

FIG. 2 shows an example view of the subject-facing surface 112 of the coupling body 102 when packaged in accordance with certain embodiments described herein. FIG. 2 shows a seal 228 and a package 236. The seal 228 is coupled to the subject-facing surface 112 of the middle portion 106 such that the seal 228 covers the openings 116 in the subject-facing surface 112 of the middle portion 106 (not visible in FIG. 2). The seal 228 may therefore be configured to prevent ultrasound gel from exiting the coupling body 102 through the openings 116 before use. The seal 228 may include adhesive for coupling to the subject-facing surface 112 of the coupling body 102, and the adhesive may be reversible such that the seal 228 may be removed from the coupling body 102 prior to use of the coupling body 102. In some embodiments, the adhesive may include pastes, stick or semi-sticky gels, non-water soluble polymers, hydrophobic substances, and/or tacky substances. In some embodiments, the adhesive may be a pressure-sensitive adhesive (PSA) including, for example, acrylics, butyl rubbers, natural rubber, nitrile rubber, ethylene-vinyl acetate, silicone rubbers, and styrene-rubber block copolymers. The seal 228 further includes a tab 234 configured to facilitate removal of the seal 228 from the coupling body 102 by a user. The coupling body 102 and the seal 228 are surrounded and sealed by a package 236. The package 236 may include, for example, polyethylene, polyethylene terephthalate, aluminum foil, metallized polyethylene, polypropylene, polyamide, and/or nylon. The coupling body 102 and the interior of the package 236 may be sterile prior to opening of the package 236.

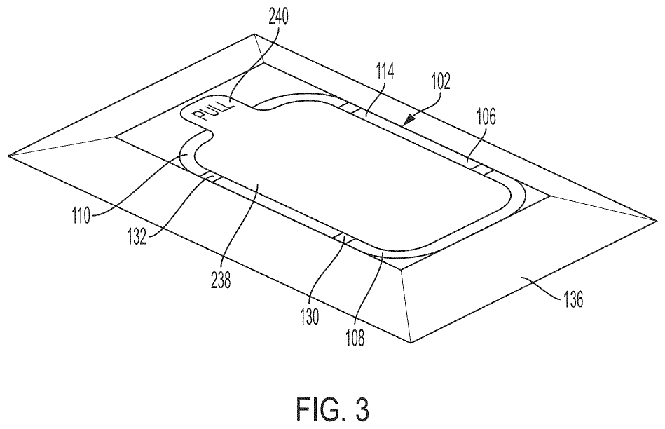

FIG. 3 shows an example view of the device-facing surface 114 of the coupling body 102 when packaged in accordance with certain embodiments described herein. FIG. 3 shows a seal 238. The seal 238 is coupled to the device-facing surface 114 of the first end portion 108, the middle portion 106, and the second end portion 110 such that the seal 238 covers the adhesive on the device-facing surface of the first end portion 108 and the second end portion 110 and the openings on the device-facing surface 114 of the middle portion 106 (not visible in FIG. 3). The seal 238 may therefore be configured to prevent ultrasound gel from exiting the coupling body 102 through the openings in the device-facing surface 114 before use, and to prevent the adhesive from being damaged (e.g., adhering to other items, losing stickiness, etc.) prior to use. The seal 238 may include adhesive for coupling to the device-facing surface 114 of the coupling body 102, and the adhesive may be reversible such that the seal 238 may be removed from the coupling body 102 prior to use of the coupling body 102. The seal 238 further includes a tab 240 configured to facilitate removal of the seal 238 from the coupling body 102 by a user.

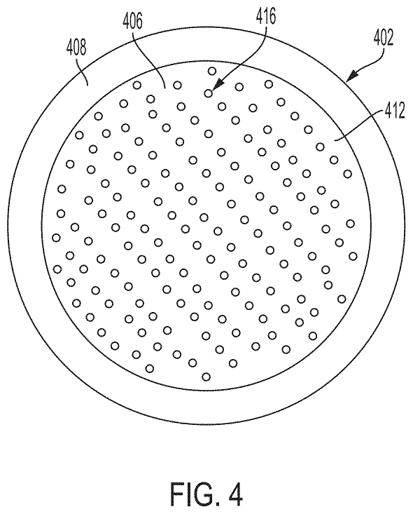

FIG. 4 shows an example view of a subject-facing surface 412 of a coupling body 402 configured to adhere to a subject in accordance with certain embodiments described herein. The coupling body 402 includes a middle portion 406 and an outer portion 408. The subject-facing surface 412 of the middle portion 406 of the coupling body 402 includes openings 416. The subject-facing surface 412 of one or both of the middle portion 406 and the outer portion 408 includes adhesive.

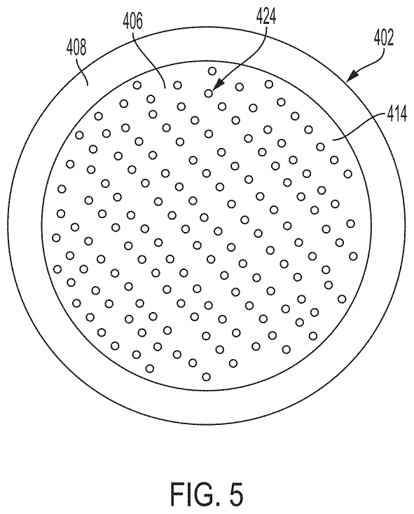

FIG. 5 shows an example view of a device-facing surface 414 of the coupling body 402 in accordance with certain embodiments described herein. The device-facing surface 414 of the coupling body 402 is on the opposite side of the coupling body 402 as the subject-facing surface 412. The device-facing surface 414 of middle portion 406 of the coupling body 402 includes openings 424. In some embodiments, the device-facing surface 414 of one or both of the outer portion 408 and the middle portion 406 of the coupling body 402 may include adhesive. In other embodiments, the device-facing surface 414 of the outer portion 408 and the middle portion 406 of the coupling body 402 may not include adhesive.

The subject-facing surface 412 of the coupling body 402 is configured to adhere to a subject during ultrasound imaging with an ultrasound device. In particular, the adhesive on the subject-facing surface 412 of the outer portion 408 and/or the middle portion 406 of the coupling body 402 is configured to adhere to the subject at an anatomical location of interest for the ultrasound imaging. When the subject-facing surface 412 of the coupling body 402 adheres to the subject, the device-facing surface 414 of the coupling body 402 faces away from the subject. A user may place an ultrasound device in contact with the device-facing surface 414 of the coupling body 402 during imaging, and the coupling body 402 is configured to interpose between the ultrasound device and the subject during imaging. The coupling body 402 may be formed of any acoustically transparent material such that, when the coupling body 402 interposes between the ultrasound device and the subject, the coupling body 402 establishes acceptable impedance matching coupling for ultrasound signal transmission and reception between the ultrasound device and the subject. The coupling body 402 may include, for example, hydrogel or polypropylene.

Adhering the coupling body 402 to a subject may be useful, for example, when ultrasound imaging of a specific anatomical feature must be performed over an extended period of time. A user may search, with an ultrasound device, for a location on the subject where the anatomical feature can be imaged, and once the location is found, the user may adhere the coupling body 402 to that location. Henceforth, the coupling body 402 may serve as a landmark for where the ultrasound device should be placed to image the anatomical feature of interest.

The coupling body 402 contains ultrasound gel in one or more internal reservoirs. The openings 416 on the subject-facing surface 412 of the middle portion 406 extend from subject-facing surface 412 to the one or more internal reservoirs. Similarly, the openings 424 on the device-facing surface 414 of the middle portion 406 extend from device-facing surface 414 to the one or more internal reservoirs. Accordingly, ultrasound gel may flow from the one or more internal reservoirs, out of the coupling body, and onto the subject (through the openings 416) and onto the ultrasound device (through the openings 424) when the ultrasound device is pressed against the device-facing surface 414 of the middle portion 406. The ultrasound gel may be an acoustically transparent material and may fill air gaps between the coupling body 402 and the subject and/or the ultrasound device during imaging, thereby improving impedance matching coupling for ultrasound signal transmission and reception. For further description of the openings, the ultrasound gel, adhesives, and other features of coupling bodies, see the discussion with reference to FIG. 1. As will be discussed below, in some embodiments the coupling body 402 may not contain one or more internal reservoirs for ultrasound gel, and may lack the openings 416 and/or 424.

While the coupling body 402 is shown as being substantially circular, in other embodiments the coupling body 402 may have different shapes, such as a substantially rectangular shape. The coupling body 402 may be shaped/sized to conform to a particular ultrasound device, or to conform to a particular body area (i.e., chest, abdomen, etc.), or to a particular size patient.

The portions of the subject-facing surface of the coupling body 402 having adhesive on them may be chosen for ease of use and sufficient adhesion. For example, too little adhesive may result in poor adhesion, while too much adhesive may be difficult or tedious for a user to adhere to the subject. In some embodiments, the adhesive may be on the entire subject-facing surface 412. In other embodiments, the adhesive may only be on a portion of the subject-facing surface 412. For example, the adhesive may only be on the subject-facing surface 412 of the outer portion 408 of the coupling body 402. The adhesive may include pastes, stick or semi-sticky gels, non-water soluble polymers, hydrophobic substances, and/or tacky substances. The adhesive may be reversible, in other words, the coupling body 402 may be removed from the subject after having been adhered to the subject.

In FIGS. 4-5, the openings 416 and the openings 424 are positioned across substantially all portions of the subject-facing surface 412 of the coupling body 402 and the device-facing surface 414 of the coupling body 402. When an ultrasound device contacts the coupling body 402, the openings 416 and the openings 424 may face the ultrasound device's acoustic lens and may therefore be positioned along the path of acoustic waves transmitted through the acoustic lens. This may help the user to dispense ultrasound gel in the direct path of the acoustic waves, where acoustic coupling is needed. However, in other embodiments, the openings 416 and the openings 424 may not be positioned on portions of the subject-facing surface 412 and device-facing surface 414 of the coupling body 402 that are along the path of acoustic waves transmitted through the acoustic lens, but may be positioned on outer portions of the subject-facing surface 412 and device-facing surface 414 of the coupling body 402 that, when an ultrasound device contacts the coupling body 402, are adjacent to but not facing the ultrasound device's acoustic lens (i.e., adjacent but outside of the periphery of the acoustic lens). This may help to reduce interference in propagation of the acoustic lens caused by the openings 416 and the openings 424. In such embodiments, once ultrasound gel is dispensed through the openings 416 and the openings 424 near the periphery of the acoustic lens, a user may use the ultrasound device to wipe the ultrasound gel into a desired position (e.g., between the subject and the acoustic lens).

As discussed above, the diameter of openings in a coupling body may be selected based on how long the coupling body needs to remain usable. In particular the diameter of the openings may be selected to be small enough such that the ultrasound gel does not dehydrate at an unacceptable rate. Subject to this constraint, the diameter of the openings may also be selected to be as large as possible to facilitate release of ultrasound gel through the openings without requiring application of unacceptably large force. For example, certain embodiments of coupling bodies configured to adhere to an ultrasound device for a single imaging device may have a larger diameter for the openings than embodiments of coupling bodies configured to adhere to a patient and an ultrasound patch device for a prolonged period of time (e.g., hours, days, or weeks). As also discussed above, certain embodiments of coupling bodies may have openings across substantially all of its subject- and device-facing surfaces, while certain embodiments of coupling bodies may have openings only at outer portions of the subject- and device-facing surfaces. Accordingly, embodiments of coupling bodies may include, as non-limiting examples: 1. Coupling bodies with relatively large diameter openings across substantially all of their subject- and device-facing surfaces 2. Coupling bodies with relatively small diameter openings across substantially all of their subject- and device-facing surfaces 3. Coupling bodies with relatively large diameter openings on outer portions of their subject- and device-facing surfaces and 4. Coupling bodies with relatively small diameter openings on outer portions of their subject- and device-facing surfaces. The diameter of the openings may be, for example, 100 microns, 200 microns, 300 microns, 400 microns, or 500 microns, where the relatively large diameter openings are larger than the relatively small diameter openings,

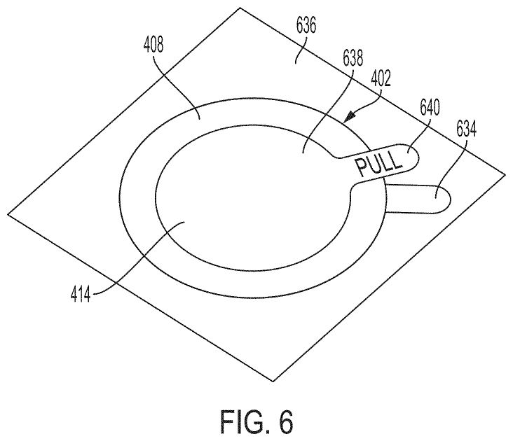

FIG. 6 shows an example view of the device-facing surface 414 of the coupling body 402 when packaged in accordance with certain embodiments described herein. FIG. 6 shows a seal 638 and a package 636. The seal 638 is coupled to the device-facing surface 414 of the middle portion 406 (not visible) of the coupling body 402 such that the seal 638 covers the openings 424 (not visible). The seal 638 may therefore be configured to prevent ultrasound gel from exiting the coupling body 402 through the openings 424 before use. The seal 638 may include adhesive for coupling to the device-facing surface 414 of the coupling body 402, and the adhesive may be reversible such that the seal 638 may be removed from the coupling body 402 prior to use of the coupling body 402. In some embodiments, the adhesive may include pastes, stick or semi-sticky gels, non-water soluble polymers, hydrophobic substances, and/or tacky substances. In some embodiments, the adhesive may be pressure-sensitive adhesive (PSA) including, for example, acrylics, butyl rubbers, natural rubber, nitrile rubber, ethylene-vinyl acetate, silicone rubbers, and styrene-rubber block copolymers. The seal 638 further includes a tab 640 configured to facilitate removal of the seal 638 from the coupling body 402 by a user. A tab 634 configured to facilitate removal of a seal 628 (not visible) from the subject-facing surface of the coupling body 402 is also visible in FIG. 6. The coupling body 402, the seal 638, and the seal 628 are surrounded and sealed by a package 636. The package 636 may include, for example, polyethylene, polyethylene terephthalate, aluminum foil, metallized polyethylene, polypropylene, polyamide, and/or nylon, and the coupling body 402 and interior of the package 636 may be sterile prior to opening of the package 636.

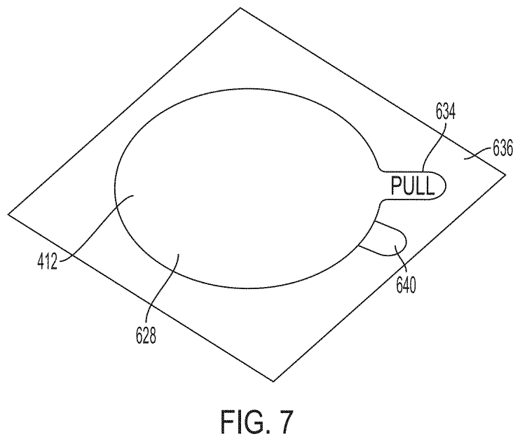

FIG. 7 shows an example view of the subject-facing surface 412 of the coupling body 402 when packaged in accordance with certain embodiments described herein. FIG. 7 shows the seal 628. The seal 628 is coupled to the subject-facing surface 412 of the middle portion 406 (not visible) and the outer portion 408 (not visible) of the coupling body 402 such that the seal 628 covers the openings 416 (not visible) and the adhesive on the outer portion 408 and/or the middle portion 406. The seal 628 may therefore be configured to prevent ultrasound gel from exiting the coupling body 402 through the openings 416 before use, and to prevent the adhesive from being damaged (e.g., adhering to other items, losing stickiness, etc.) prior to use. The seal 628 may include adhesive for coupling to the subject-facing surface 412 of the coupling body 402, and the adhesive may be reversible such that the seal 628 may be removed from the coupling body 402 prior to use of the coupling body 402. The seal 628 further includes the tab 634 configured to facilitate removal of the seal 628 from the coupling body 402 by a user.

As discussed above, in some embodiments the device-facing surface 414 of one or both of the outer portion 408 and the middle portion 406 of the coupling body 402 may include adhesive. This may be useful, for example, when adhering an ultrasound device, such as an ultrasound patch, to a subject. In particular, an ultrasound patch may be adhered to the adhesive on the device-facing surface 414 of the coupling body 402 while the coupling body 402 is adhered to the subject by the adhesive on the subject-facing surface 412. Henceforth, the ultrasound patch may remain in place on the subject, capable of imaging continuously and/or periodically. In some embodiments, a user may press the ultrasound patch that is adhered to the subject against the subject in order to improve acoustic coupling between the ultrasound patch and the subject when the ultrasound patch is collecting ultrasound data.

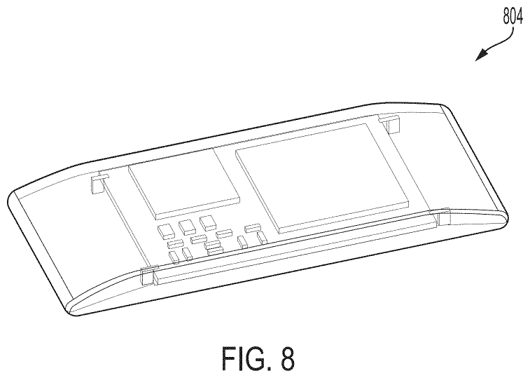

FIG. 8 shows an example of an ultrasound patch 804 in accordance with certain embodiments described herein. The patch 804 may be configured to transmit, wirelessly for example, data collected by the patch 804 to one or more external devices (not shown) for further processing. For purposes of illustration, a top housing of the patch 804 is depicted in a transparent manner to depict exemplary locations of various internal components of the patch 804.

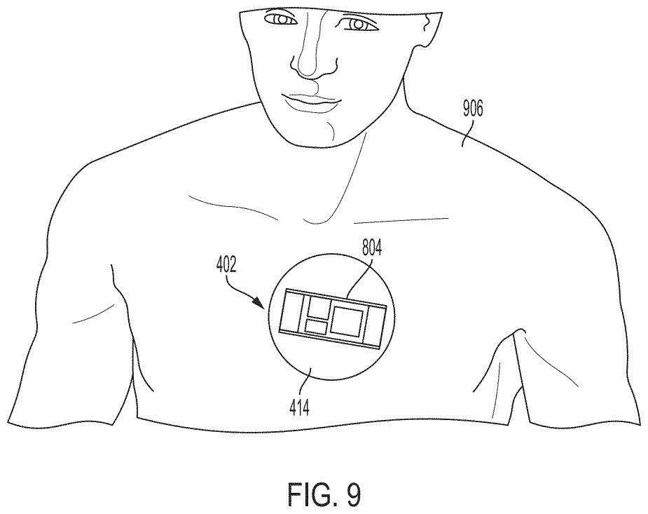

FIG. 9 shows an example of the patch 804 coupled to a patient 806 in accordance with certain embodiments described herein. The patch 804 is coupled to the patient 906 by adhering to adhesive on the device-facing surface 414 of the coupling body 402, which in turn is coupled to the patient by adhesive on the subject-facing surface 412 of the coupling body 402.

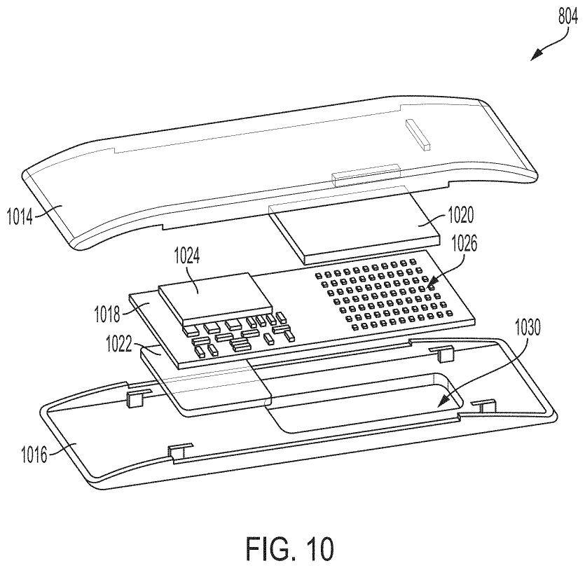

FIG. 10 shows an example exploded view of the patch 804 in accordance with certain embodiments described herein. As particularly illustrated in FIG. 10, the patch 804 includes an upper housing 1014, a lower housing 1016, and a circuit board 1018. The circuit board 1018 may be configured to support various components, such as a heat sink 1020, a battery 1022, and communications circuitry 1024. In one embodiment, the communication circuitry 1024 includes one or more short- or long-range communication platform. Exemplary short-range communication platforms include, Bluetooth (BT), Bluetooth Low Energy (BLE), and Near-Field Communication (NFC). Exemplary long-range communication platforms include, Wi-Fi and Cellular. While not shown, the communication platform may include front-end radio, antenna and other processing circuitry configured to communicate radio signal to an auxiliary device (not shown). The radio signal may include ultrasound imaging information obtained by the patch 804.

In an exemplary embodiment, the communication circuitry 1024 may transmit periodic beacon signals according to IEEE 802.11 and other prevailing standards. The beacon signal may include a BLE advertisement. Upon receipt of the beacon signal or the BLE advertisement, an auxiliary device (not shown) may respond to the patch 804. That is, the response to the beacon signal may initiate a communication handshake between the patch 804 and the auxiliary device.

The auxiliary device may include a laptop, desktop, smartphone, or any other device configured for wireless communication. The auxiliary device may act as a gateway to cloud or internet communication. In an exemplary embodiment, the auxiliary device may include the patient's own smart device (e.g., smartphone) which communicatively couples to the patch 804 and periodically receives ultrasound information from the patch 804. The auxiliary device may then communicate the received ultrasound information to external sources.

The circuit board 1018 may include processing circuitry, including one or more controllers to direct communication through communication circuitry 1024. For example, the circuit board 1018 may engage the communication circuitry 1024 periodically or on as-needed basis to communicate information with one or more auxiliary devices. Ultrasound information may include signals and information defining an ultrasound image captured by the patch 804. Ultrasound information may also include control parameters communicated from the auxiliary device to the patch 804. The control parameters may dictate the scope of the ultrasound image to be obtained by the patch 804.

In one embodiment, the auxiliary device may store ultrasound information received from the patch 804. In another embodiment, the auxiliary device may relay ultrasound information received from the patch 804 to another station. For example, the auxiliary device may use Wi-Fi to communicate the ultrasound information received from patch 804 to a cloud-based server. The cloud-based server may be a hospital server or a server accessible to the physician directing ultrasound imaging. In another exemplary embodiment, the patch 804 may send sufficient ultrasound information to the auxiliary device such that the auxiliary device may construct an ultrasound image therefrom. In this manner, communication bandwidth and power consumption may be minimized at the patch 804.

In still another embodiment, the auxiliary device may engage the patch 804 through radio communication (i.e., through the communication circuitry 1024) to actively direct operation of the patch 804. For example, the auxiliary device may direct the patch 804 to produce ultrasound images of the patient at periodic intervals. The auxiliary device may direct the depth of the ultrasound images taken by the patch 804. In still another example, the auxiliary device may control the manner of operation of the patch so as to preserve power consumption at the battery 1022. Upon receipt of ultrasound information from the patch 804, the auxiliary device may operate to cease imaging, increase imaging rate, or communicate an alarm to the patient or to a third party (e.g., a physician or emergency personnel). As shown in FIG. 10, a plurality of through vias 1026 (e.g., copper) may be used for a thermal connection between the heat sink 1020 and one or more image reconstruction chips (e.g., CMOS) (not shown in FIG. 10). The lower housing 1016 is configured to be adhered to the coupling body 402 and includes an opening 1030 which, when the patch lower housing 1016 is adhered to a coupling body 402, opens to the coupling body 402.

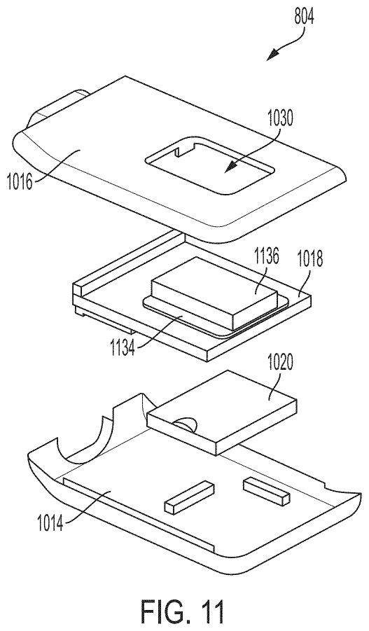

FIG. 11 shows an example “bottom up” exploded view of the patch 804 in accordance with certain embodiments described herein. FIG. 11 illustrates the location of ultrasonic transducers and integrated CMOS chip (generally indicated by 1134) on the circuit board 1018. An acoustic lens 1136 mounted over the transducers/CMOS 1134 is configured to protrude through the opening 1030 to make contact with the device-facing surface 414 of the coupling body 402.

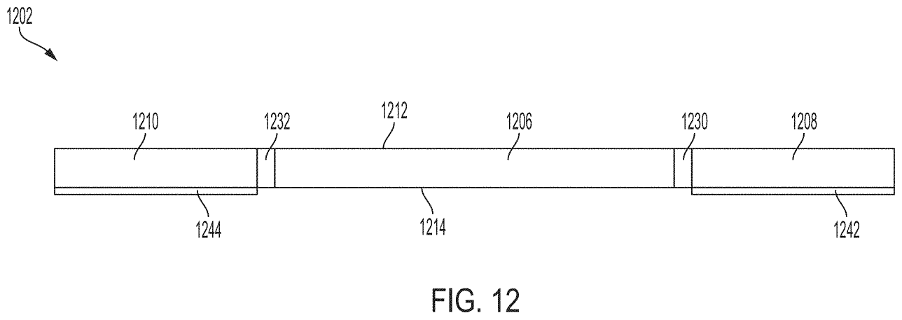

FIGS. 12-19 show example cross-sections of coupling bodies. It will be appreciated that aspects and features of the coupling bodies shown in FIGS. 12-19 may apply to the coupling bodies 102 and 402, and vice versa.

FIG. 12 shows an example cross-section of a coupling body 1202 in accordance with certain embodiments described herein. The coupling body 1202 includes a middle portion 1206, a first end portion 1208, a second end portion 1210, a first flexible member 1230, a second flexible member 1232, a first surface 1212, and a second surface 1214. The first flexible member 1230 is coupled between the middle portion 1206 and the first end portion 1208. The second flexible member 1232 is coupled between the middle portion 1206 and the second end portion 1210. The first end portion 1208 includes an adhesive 1242 on the second surface 1214 of the first end portion 1208. The second end portion 1210 includes an adhesive 1244 on the second surface 1214 of the second end portion 1210.

In some embodiments, the coupling body 1202 is configured to couple to an ultrasound device. In such embodiments, the second surface 1214 of the coupling body 1202 is configured to couple to the ultrasound device. In particular, the adhesive 1242 and the adhesive 1244 on the second surface 1214 of the coupling body 1202 is configured to adhere to the ultrasound device. In such embodiments, the first surface 1212 of the coupling body 1202 is configured to contact a subject during ultrasound imaging with the ultrasound device.

In some embodiments, the coupling body 1202 is configured to couple to a subject. In such embodiments, the second surface 1214 of the coupling body 1202 is configured to couple to the subject. In particular, the adhesive 1242 and the adhesive 1244 on the second surface 1214 of the coupling body 1202 is configured to adhere to the subject. In such embodiments, an ultrasound device is configured to contact the first surface 1212 of the coupling body 1202 during ultrasound imaging.

The coupling body 1202 (in particular, the middle portion 1206, the first end portion 1208, and the second end portion 1210) may be formed of any acoustically transparent material such that, when the coupling body 1202 interposes between the ultrasound device and the subject, the coupling body 1202 establishes acceptable impedance matching coupling for ultrasound signal transmission and reception. In some embodiments, the coupling body 1202 includes a material having a characteristic acoustic impedance differing from that of water by at most 6×10⁵ N·s/m³ (e.g., by at most 5×10⁵ N·s/m³). The characteristic acoustic impedance of water is approximately 1.494×10⁶ N·s/m³, and accordingly the coupling body 102 may include as non-limiting examples polypropylene, polydimethylsiloxane (PDMS), and/or agar (which have characteristic acoustic impedances of approximately 2.002×10⁶ N·s/m³, 1.1×10⁶ N·s/m³, and 1.52-1.76×10⁶ N·s/m³, respectively). In embodiments in which the coupling body 1202 includes PDMS and/or agar, to improve the mechanical durability of the coupling body 1202, the PDMS and/or agar could be cured around a mechanically reinforcing screen or mesh. The screen or mesh could be made of nylon or other biologically inert plastic with large openings to allow an acceptable level of ultrasound penetration and thin diameter filaments for reinforcement.

In some embodiments, the coupling body 1202 may include a hydrogel. For example, the hydrogel may be formed of a hydrophilic monomer/polymer, crosslinking agent, and catalyst. The hydrophilic monomer/polymer may include, for example, methacrylic acid, salts of methacrylic acid, esters of methacrylic acid, salts and acids of esters of methacrylic acid, amides of methacrylic acid, N-alkyl amides of methacrylic acid, salts and acids of N-alkyl amides of methacrylic acid, N-vinylpyrrolidone, acrylamide, acrylamide derivatives, methacrylamide, methacrylamide derivatives, acrylamide, N-isopropylacrylamide, 2-hydroxyethyl methacrylate (HEMA), 2-hydroxypropyl methacrylate, acrylic acid, 2-acrylamido-2-methyl-1-propanesulfonic acid, 3-sulfopropyl acrylate potassium salt, 2-(acryloyloxy)ethyl]trimethylammonium methyl sulfate and its inorganic salts, 2-(methacryloyloxy)ethyl]trimethylammonium methyl sulfate and its inorganic salts, or any combination thereof. The crosslinking agent may include, for example, ethylene glycol dimethacrylate, ethylene glycol diacrylate, poly(ethylene glycol)dimethacrylate, poly(ethylene glycol)diacrylate, poly(ethylene glycol)diacrylamide, N,N′-methylenebisacrylamide, piperazine diacrylamide, glutaraldehyde, epichlorohydrin, crosslinking agents containing 1,2-diol structures, crosslinking agents containing functionalized peptides, and crosslinking agents containing proteins. The catalyst may include, for example, benzoyl peroxide, ammonium persulfate, sodium bisulfite, potassium persulfate, sodium persulfate, and the potassium salt of persulfate. In some embodiments, the coupling may further include a water-soluble polymer, such as polyvinylpyrrolidone, polyethylene glycol, or polyethylene oxide, in addition to a hydrogel. In some embodiments, the coupling body 102 may include room-temperature-vulcanizing silicone, a Sylgard® silicone, or polystyrene.

The portions of the coupling body 1202 having adhesive on them may be chosen for ease of use and sufficient adhesion. For example, too little adhesive may result in poor adhesion, while too much adhesive may be difficult or tedious for a user to adhere to the ultrasound device. In some embodiments, the adhesive 1242 and the adhesive 1244 may extend over the entire second surface 1214 of the first end portion 1208 and the second end portion 1210, respectively. In other embodiments, the adhesive may only be on a portion of the entire second surface 1214 of the first end portion 1208 and the second end portion 1210. For example, the adhesive 1242 and the adhesive 1244 may be on a perimeter portion of the second surface 1214 of the first end portion 1208 and the second end portion 1210. The adhesive 1242 and the adhesive 1244 may include pastes, stick or semi-sticky gels, non-water soluble polymers, hydrophobic substances, and/or tacky substances. The adhesive may be reversible, in other words, the coupling body 1202 may be removed from the ultrasound device/subject after having been adhered to the ultrasound device/subject.

The first flexible member 1230 is coupled between the middle portion 1206 of the coupling body 1202 and the first end portion 1208 of the coupling body 1202. The second flexible member 1232 is coupled between the middle portion 1206 of the coupling body 1202 and the second end portion 1210 of the coupling body 1202. The first flexible member 1230 and the second flexible member 1232 may be configured as hinges. In particular, the first flexible member 1230 may be configured to enable the first end portion 1208 to rotate about the first flexible member 1230 substantially independently of the middle portion 1206. Similarly, the second flexible member may be configured to enable the second end portion 1210 to rotate about the second flexible member 1232 substantially independently of the middle portion 1206. Therefore, the first end portion 1208 and the second end portion 1210 may rotate with respect to the middle portion 1206 to assume a shape that conforms to the ultrasound device. This may be helpful for ensuring tight coupling of the coupling body 1202 to the ultrasound device and for enabling use of the coupling body 1202 with a variety of ultrasound devices having different shapes (e.g., linear, phased array, curvilinear, transesophageal, and transvaginal ultrasound devices). The first flexible member 1230 and the second flexible member 1232 may include any type of flexible material, such as elastic material, a polymer, polystyrene, or polypropylene, or aluminum foil. The elastic modulus of the flexible material may be in the range of approximately 0.15 GPa and 14.7 GPa. In some embodiments, the primary material of the coupling body 1202 itself may allow the coupling body 1202 to assume a shape that conforms to the ultrasound device, and the first flexible member 1230 and the second flexible member 1232 may not be necessary.

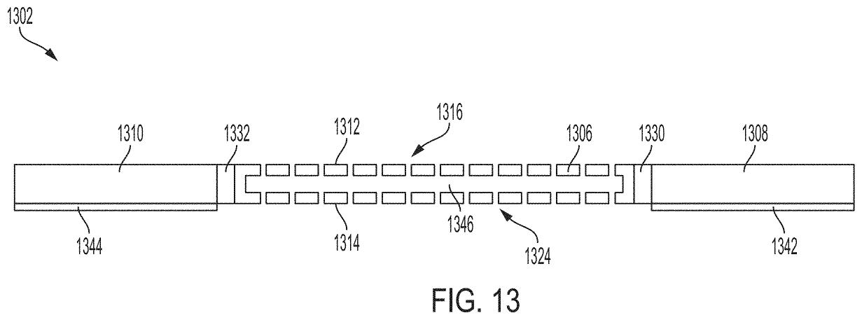

FIG. 13 shows an example cross-section of a coupling body 1302 in accordance with certain embodiments described herein. The coupling body 1302 includes a middle portion 1306, a first end portion 1308, a second end portion 1310, a first flexible member 1330, a second flexible member 1332, a first surface 1312, and a second surface 1314. The first flexible member 1330 is coupled between the middle portion 1306 and the first end portion 1308. The second flexible member 1332 is coupled between the middle portion 1306 and the second end portion 1310. The first end portion 1308 includes an adhesive 1342 on the second surface 1314 of the first end portion 1308. The second end portion 1310 includes an adhesive 1344 on the second surface 1314 of the second end portion 1310. The following description discusses differences between the coupling body 1302 and the coupling body 1202. For discussion of other aspects of the coupling body 1302 (e.g., materials), see the discussion with reference to FIG. 12. The coupling body 1302 differs from the coupling body 1202 in that the coupling body 1302 includes openings 1316 in the first surface 1312 of the middle portion 1306 and openings 1324 in the second surface 1314 of the middle portion 1306. The middle portion 1306 further includes an internal reservoir 1346 that may constitute a cavity within the middle portion 1306. The coupling body 1302 may include more reservoirs and openings outside of the plane of the cross-section shown in FIG. 13.

The internal reservoir 1346 may contain ultrasound gel. The openings 1316 on the first surface 1312 of the middle portion 1306 extend from the first surface 1312 to the internal reservoir 1346. Similarly, the openings 1324 on the second surface 1314 of the middle portion 1306 extend from the second surface 1314 to the internal reservoir 1346. Accordingly, ultrasound gel may flow from the internal reservoir 1346, out of the coupling body 1302 through the openings 1316 and the openings 1324, and onto the ultrasound device and the subject. The ultrasound gel may be an acoustically transparent material and may fill air gaps between the coupling body 1302 and the subject and/or the ultrasound device during imaging, thereby improving impedance matching coupling for ultrasound signal transmission and reception between the subject and the ultrasound device.

The openings 1316 and the openings 1324 may be configured to release ultrasound gel from the internal reservoir 1346 in response to a user pressing down on the coupling body 1302 with an ultrasound device. The diameter of the openings 1316 and the openings 1324 may be selected to be small enough (e.g., less than 500 microns) such that the user can control, based on force applied, how much ultrasound gel the openings 1316 and the openings 1324 release with acceptable precision. The diameter of the openings 1316 and the openings 1324 may be, for example, on the micron or nanometer scale. Furthermore, the coupling body 1302 may be configured to allow the user to selectively apply ultrasound gel in certain locations, for example by pushing down with the ultrasound device on certain portions of the coupling body 1302 but not others. This may help the user to apply a sufficient amount of ultrasound gel to obtain an image of acceptable quality, without applying too much ultrasound gel, which can be messy and uncomfortable. The openings 1316 and the openings 1324 may be formed in a material (e.g., a hydrogel or polypropylene) that is sufficiently rigid such that the openings 1316 and the openings 1324 do not collapse when pressure is applied to them. The pitch of the openings 1316 and the openings 1324 may be selected to be small enough (e.g. less than 5 mm) such that there are a sufficient number of openings 1316 and openings 1324 in the coupling body 1302 to release a sufficient amount of ultrasound gel for collecting images of acceptable quality. Ultrasound gel at the interface between the ultrasound device and the coupling body 1302 (whether that interface be at the first surface 1312 or the second surface 1314). This lubricious interface may help a user of the ultrasound device to expel air bubbles at the interface when the user presses down on the ultrasound device.

In some embodiments, the ultrasound gel may include one or more humectants as described herein. In some embodiments, the ultrasound gel may include preservatives, antiseptics, anti-fungals, anti-bacterials, bactericidal and bacteriostatic agents (e.g., alcohols, hydrogen peroxide, and organic or inorganic salts), and/or organic acids (e.g., citric acid, lactic acid, and acetic acid). Such substances may help to keep mold, fungus, and microbes out of the ultrasound gel by killing them or inhibiting/slowing/stalling their growth. This in turn may help to prevent infections, passing of diseases, spread of illnesses, etc.

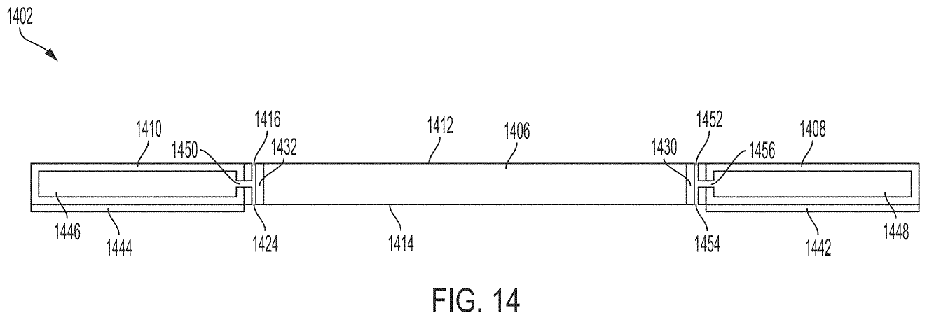

FIG. 14 shows an example cross-section of a coupling body 1402 in accordance with certain embodiments described herein. The coupling body 1402 includes a middle portion 1406, a first end portion 1408, a second end portion 1410, a first flexible member 1430, a second flexible member 1432, a first surface 1412, and a second surface 1414. The first flexible member 1430 is coupled between the middle portion 1406 and the first end portion 1408. The second flexible member 1432 is coupled between the middle portion 1406 and the second end portion 1410. The first end portion 1408 includes an adhesive 1442 on the second surface 1414 of the first end portion 1408. The second end portion 1410 includes an adhesive 1444 on the second surface 1414 of the second end portion 1410. The following description discusses differences between the coupling body 1402 and the coupling body 1202. For discussion of other aspects of the coupling body 1402 (e.g., materials), see the discussion with reference to FIG. 12. The coupling body 1402 differs from the coupling body 1202 in that the coupling body 1402 includes an internal reservoir 1446 in the second end portion 1410, an opening 1416 in the first surface 1412 of the second flexible member 1432, an opening 1424 in the second surface 1414 of the second flexible member 1432, and a channel 1450 extending from the internal reservoir 1446 to the opening 1416 and the opening 1424. Furthermore, the coupling body 1402 includes an internal reservoir 1448 in the first end portion 1408, an opening 1452 in the first surface 1412 of the first flexible member 1430, an opening 1454 in the second surface 1414 of the first flexible member 1430, and a channel 1456 extending from the internal reservoir 1448 to the opening 1452 and the opening 1454. The internal reservoir 1446 and the internal reservoir 1448 may contain ultrasound gel. Ultrasound gel may flow from the internal reservoir 1446, through the channel 1450, out of the coupling body 1402 through the opening 1416 and the opening 1424, and onto the ultrasound device and the subject. Similarly, ultrasound gel may flow from the internal reservoir 1448, through the channel 1456, out of the coupling body 1402 through the opening 1452 and the opening 1454, and onto the ultrasound device and the subject. The coupling body 1402 may include more reservoirs and openings outside of the plane of the cross-section shown in FIG. 14. For further discussion of ultrasound gel and openings, see the discussion with reference to FIG. 13.

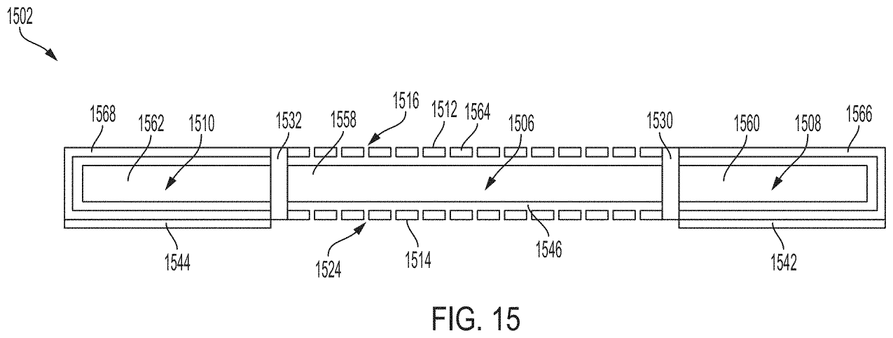

FIG. 15 shows an example cross-section of a coupling body 1502 in accordance with certain embodiments described herein. The coupling body 1502 includes a middle portion 1506, a first end portion 1508, a second end portion 1510, a first flexible member 1530, a second flexible member 1532, a first surface 1512, and a second surface 1514. The first flexible member 1530 is coupled between the middle portion 1506 and the first end portion 1508. The second flexible member 1532 is coupled between the middle portion 1506 and the second end portion 1510. The first end portion 1508 includes an adhesive 1542 on the second surface 1514 of the first end portion 1508. The second end portion 1510 includes an adhesive 1544 on the second surface 1514 of the second end portion 1510. The following description discusses differences between the coupling body 1502 and the coupling body 1202. For discussion of other aspects of the coupling body 1502 (e.g., materials), see the discussion with reference to FIG. 12. The coupling body 1502 differs from the coupling body 1202 in that the middle portion 1506 includes an inner middle portion 1558 and an outer middle portion 1564, the first end portion 1508 includes an inner first end portion 1560 and an outer first end portion 1566, and the second end portion 1510 includes an inner second end portion 1562 and an outer second end portion 1568. Together, the inner middle portion 1558, the inner first end portion 1560, and the inner second end portion 1562 may be considered an inner portion of the coupling body 1502, and the outer middle portion 1564, the outer first end portion 1566, and the outer second end portion 1568 may be considered an outer portion of the coupling body 1502, with the outer portion surrounding the inner portion. Furthermore, the coupling body 1502 includes openings 1516 in the first surface 1512 of the outer middle portion 1564 and openings 1524 in the second surface 1514 of the outer middle portion 1564. The middle portion 1506 further includes an internal reservoir 1546 disposed between the inner middle portion 1558 and the outer middle portion 1564.

The internal reservoir 1546 may contain ultrasound gel. The openings 1516 in the first surface 1512 of the outer middle portion 1564 extend from the first surface 1512 to the internal reservoir 1546. Similarly, the openings 1524 in the second surface 1514 of the outer middle portion 1564 extend from the second surface 1514 to the internal reservoir 1546. Accordingly, ultrasound gel may flow from the internal reservoir 1546, out of the coupling body 1502 through the openings 1516 and the openings 1524, and onto the ultrasound device and the subject. The coupling body 1402 may include more reservoirs and openings outside of the plane of the cross-section shown in FIG. 15. For further discussion of ultrasound gel and openings, see the discussion with reference to FIG. 13.

The inner middle portion 1558, the inner first end portion 1560, and the inner second end portion 1562 may include, for example, hydrogel material. For example, the hydrogel may be formed of a hydrophilic monomer/polymer, crosslinking agent, and catalyst. The hydrophilic monomer/polymer may include, for example, methacrylic acid, salts of methacrylic acid, esters of methacrylic acid, salts and acids of esters of methacrylic acid, amides of methacrylic acid, N-alkyl amides of methacrylic acid, salts and acids of N-alkyl amides of methacrylic acid, N-vinylpyrrolidone, acrylamide, acrylamide derivatives, methacrylamide, methacrylamide derivatives, acrylamide, N-isopropylacrylamide, 2-hydroxyethyl methacrylate (HEMA), 2-hydroxypropyl methacrylate, acrylic acid, 2-acrylamido-2-methyl-1-propanesulfonic acid, 3-sulfopropyl acrylate potassium salt, 2-(acryloyloxy)ethyl]trimethylammonium methyl sulfate and its inorganic salts, 2-(methacryloyloxy)ethyl]trimethylammonium methyl sulfate and its inorganic salts, or any combination thereof. The crosslinking agent may include, for example, ethylene glycol dimethacrylate, ethylene glycol diacrylate, poly(ethylene glycol)dimethacrylate, poly(ethylene glycol)diacrylate, poly(ethylene glycol)diacrylamide, N,N′-methylenebisacrylamide, piperazine diacrylamide, glutaraldehyde, epichlorohydrin, crosslinking agents containing 1,2-diol structures, crosslinking agents containing functionalized peptides, and crosslinking agents containing proteins. The catalyst may include, for example, benzoyl peroxide, ammonium persulfate, sodium bisulfite, potassium persulfate, sodium persulfate, and the potassium salt of persulfate. In some embodiments, the coupling body 102 may further include a water-soluble polymer, such as polyvinylpyrrolidone, polyethylene glycol, or polyethylene oxide, in addition to a hydrogel.

The outer middle portion 1564, the outer first end portion 1566, and the outer second end portion 1568 may include, for example, polypropylene, polydimethylsiloxane (PDMS), agar, room-temperature-vulcanizing (RTV) silicone, a Sylgard® silicone, or polystyrene. In some embodiments, the inner middle portion 1558, the inner first end portion 1560, the inner second end portion 1562, the outer middle portion 1564, the outer first end portion 1566, and the outer second end portion 1568 may include any material having a characteristic acoustic impedance differing from that of water by at most 6×10⁵ N·s/m³ (e.g., by at most 5×10⁵ N·s/m³).

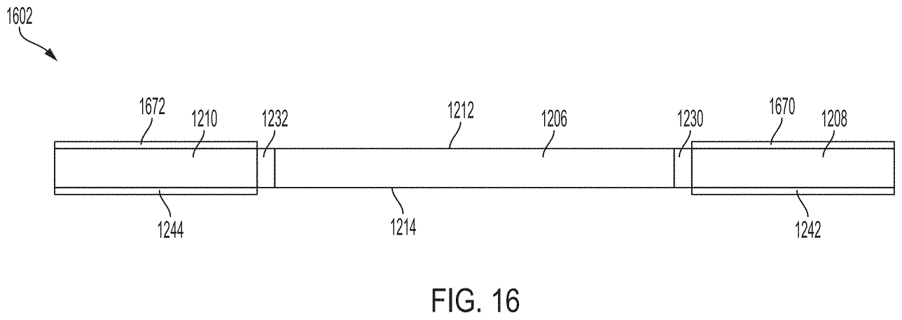

FIG. 16 shows an example cross-section of a coupling body 1602 in accordance with certain embodiments described herein. The following description discusses differences between the coupling body 1602 and the coupling body 1202. For discussion of other aspects of the coupling body 1602, see the discussion with reference to FIG. 12. The coupling body 1602 differs from the coupling body 1202 in that the coupling body 1602 includes an adhesive 1670 on the first surface 1212 of the first end portion 1208 and an adhesive 1672 on the first surface 1210 of the second end portion 1210. Accordingly, the first surface 1212 may adhere to a subject through the adhesive 1670 and the adhesive 1672 while the second surface 1214 may adhere to an ultrasound patch through the adhesive 1242 and the adhesive 1244 (or alternatively, the first surface 1212 may adhere to an ultrasound patch and the second surface 1214 may adhere to as subject).

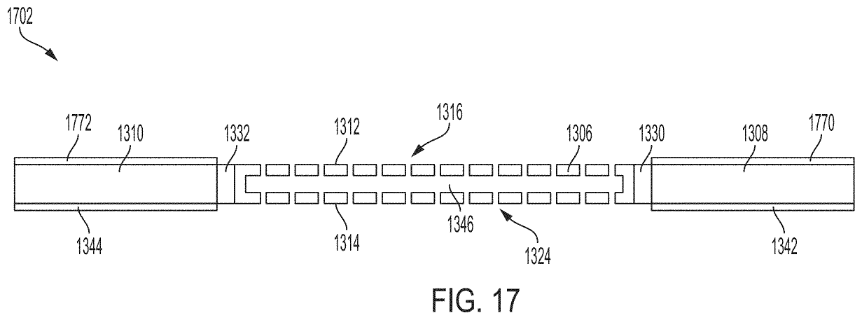

FIG. 17 shows an example cross-section of a coupling body 1702 in accordance with certain embodiments described herein. The following description discusses differences between the coupling body 1702 and the coupling body 1302. For discussion of other aspects of the coupling body 1702 (e.g., materials), see the discussion with reference to FIG. 13. The coupling body 1702 differs from the coupling body 1302 in that the coupling body 1702 includes an adhesive 1770 on the first surface 1312 of the first end portion 1308 and an adhesive 1772 on the first surface 1312 of the second end portion 1310. Accordingly, the first surface 1312 may adhere to a subject through the adhesive 1770 and the adhesive 1772 while the second surface 1314 may adhere to an ultrasound patch through the adhesive 1342 and the adhesive 1344 (or alternatively, the first surface 1312 may adhere to an ultrasound patch and the second surface 1314 may adhere to as subject).

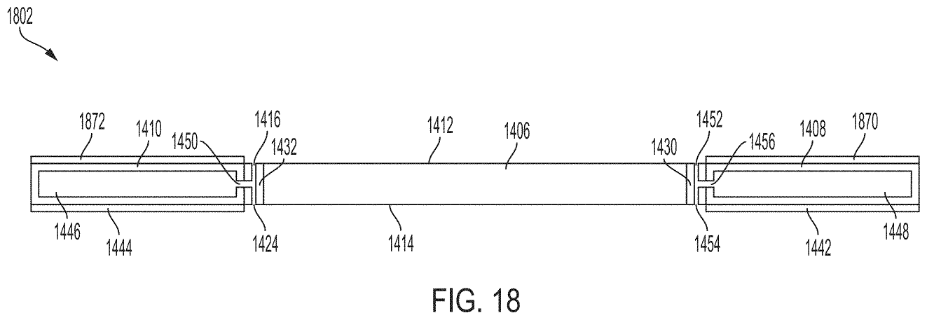

FIG. 18 shows an example cross-section of a coupling body 1802 in accordance with certain embodiments described herein. The following description discusses differences between the coupling body 1802 and the coupling body 1402. For discussion of other aspects of the coupling body 1802 (e.g., materials), see the discussion with reference to FIG. 14. The coupling body 1802 differs from the coupling body 1402 in that the coupling body 1802 includes an adhesive 1870 on the first surface 1412 of the first end portion 1408 and an adhesive 1872 on the first surface 1412 of the second end portion 1410. Accordingly, the first surface 1412 may adhere to a subject through the adhesive 1870 and the adhesive 1872 while the second surface 1414 may adhere to an ultrasound patch through the adhesive 1442 and the adhesive 1444 (or alternatively, the first surface 1412 may adhere to an ultrasound patch and the second surface 1414 may adhere to as subject).

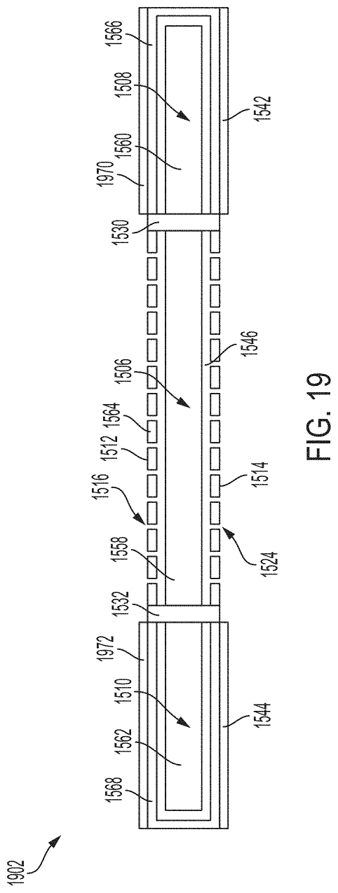

FIG. 19 shows an example cross-section of a coupling body 1902 in accordance with certain embodiments described herein. The following description discusses differences between the coupling body 1902 and the coupling body 1502. For discussion of other aspects of the coupling body 1902 (e.g., materials), see the discussion with reference to FIG. 15. The coupling body 1902 differs from the coupling body 1502 in that the coupling body 1902 includes an adhesive 1970 on the first surface 1512 of the first end portion 1508 and an adhesive 1972 on the first surface 1512 of the second end portion 1510. Accordingly, the first surface 1512 may adhere to a subject through the adhesive 1970 and the adhesive 1972 while the second surface 1514 may adhere to an ultrasound patch through the adhesive 1542 and the adhesive 1544 (or alternatively, the first surface 1512 may adhere to an ultrasound patch and the second surface 1514 may adhere to as subject).

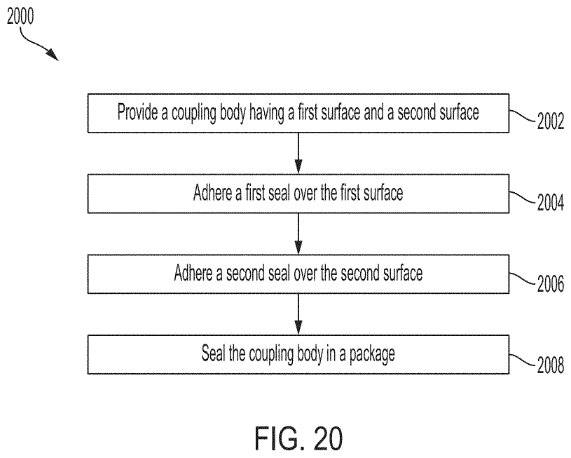

FIG. 20 shows an example process 2000 for packaging a coupling body. The process 2000 may be performed by, for example, a machine or a person.

In act 2002, a coupling body is provided. The coupling body includes a first surface and a second surface. In some embodiments, the coupling body may be configured to couple to an ultrasound device. In such embodiments, the second surface of the coupling body may be configured to couple to the ultrasound device by adhesive on the second surface. In such embodiments, the first surface of the coupling body may be configured to contact a subject during ultrasound imaging with the ultrasound device. The first surface and the second surface may further include openings, and the coupling body may include an internal reservoir configured to contain ultrasound gel, and the openings may extend from the internal reservoir to their respective surfaces.

In some embodiments, the coupling body may be configured to couple to a subject. In such embodiments, the second surface of the coupling body may be configured to couple to the subject by adhesive on the second surface. In such embodiments, an ultrasound device may be configured to contact the first surface of the coupling body during ultrasound imaging. The first surface and the second surface may further include openings, the coupling body may include an internal reservoir configured to contain ultrasound gel, and the openings may extend from the internal reservoir to their respective surfaces.

In some embodiments, the coupling body may be configured to couple to a subject, and an ultrasound patch may be configured to couple to the coupling body. In such embodiments, the second surface of the coupling body may be configured to couple to the subject by adhesive on the second surface, and the first surface may be configured to couple to the ultrasound patch by adhesive on the first surface. The first surface and the second surface may further include openings, the coupling body may include an internal reservoir configured to contain ultrasound gel, and the openings may extend from the internal reservoir to their respective surfaces. For further discussion of coupling bodies, openings, internal reservoirs, and ultrasound gel, see the discussion with reference to FIGS. 1-7 and 12-19.

In act 2004, a first seal is adhered over the first surface of the coupling body. In embodiments in the which the first surface includes openings and the coupling body includes an internal reservoir containing ultrasound gel, the first seal may be adhered over the openings on the first surface, and may therefore be configured to help prevent ultrasound gel from exiting the coupling body through the openings on the first surface before use. In embodiments in which the first surface includes adhesive, the first seal may be adhered over the adhesive on the first surface, and may therefore be configured to help prevent the adhesive from being damaged (e.g., adhering to other items, losing stickiness, etc.) prior to use. The first seal may include adhesive for coupling to the first surface of the coupling body, and the adhesive may be reversible such that the first seal may be removed from the coupling body prior to use of the coupling body. The first seal may further include a tab configured to facilitate removal of the first seal from the coupling body by a user.

In act 2006, a second seal is adhered over the openings and the adhesive on the second surface of the coupling body. In embodiments in which the second surface includes openings and adhesive and the coupling body includes an internal reservoir containing ultrasound gel, the second seal may be adhered over the openings and adhesive on the second surface, and may therefore be configured to help prevent ultrasound gel from exiting the coupling body through the openings in the second surface before use, and to prevent the adhesive from being damaged (e.g., adhering to other items, losing stickiness, etc.) prior to use. The second seal may include adhesive for coupling to the second surface of the coupling body, and the adhesive may be reversible such that the second seal may be removed from the coupling body prior to use of the coupling body. The second seal may further include a tab configured to facilitate removal of the second seal from the coupling body by a user.

In act 2008, the coupling body may be sealed in a package. For example, the coupling body may be surrounded by the package. The package may include, for example, plastic material or foil material. The coupling body and the interior of the package may be sterile prior to opening of the package.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A coupling body for acoustically coupling an ultrasound device to a subject, comprising:
a first surface configured to couple to the ultrasound device;
a second surface configured to contact the subject;
a reservoir internal to the coupling body; and
a plurality of openings extending between the reservoir and one or both of the first surface and the second surface.

2. The coupling body of claim 1, wherein the reservoir contains ultrasound gel.

3. The coupling body of claim 2, wherein the coupling body is configured to deposit the ultrasound gel through the plurality of openings from the reservoir to one or both of the first surface and the second surface in response to force applied to the body.

4. The coupling body of claim 3, wherein the ultrasound gel includes a humectant.

5. The coupling body of claim 1, wherein each of the plurality of openings is between approximately 100 microns and 500 microns in diameter and wherein the plurality of openings have a pitch of between approximately 1 mm and 5 mm.

6. The coupling body of claim 1, wherein:
the coupling body includes an inner portion and an outer portion surrounding the inner portion;
the first surface and the second surface are on the outer portion of the coupling body; and
the reservoir is disposed between the inner portion and the outer portion and wherein the inner portion comprises a hydrogel material and wherein the outer portion comprises polypropylene.

7. The coupling body of claim 1, further comprising an adhesive coupled to a subpart of the first surface of the coupling body and wherein an area of the subpart of the first surface is substantially less than an area of the first surface, the subpart including a perimeter portion of the first surface of the coupling body.

8. The coupling body of claim 7, wherein:
the coupling body is substantially rectangular;
the coupling body comprises:
a middle portion;
a first end portion; and
a second end portion; and
the adhesive is coupled to at least one of the first end portion and the second end portion and not the middle portion and wherein the middle portion, the first end portion, and the second end portion have substantially a same length.

9. The coupling body of claim 7, wherein the adhesive is configured to couple to a portion of the ultrasound device that does not include an acoustic lens of the ultrasound device.

10. The coupling body of claim 1, further comprising:
a middle portion;
a first end portion;
a second end portion;
a first flexible member coupled between the middle portion and the first end portion; and
a second flexible member coupled between the middle portion and the second end portion;
wherein the first flexible member and the second flexible member include a flexible material having an elastic modulus between approximately 0.15 GPa and 14.7 GPa.

11. The coupling body of claim 10, wherein the first flexible member and the second flexible member comprise hinges, the first flexible member and the second flexible member each include at least one of the plurality of openings and wherein the adhesive is coupled to at least one of the first end portion and the second end portion and not the middle portion.

12. A method of packaging an ultrasound coupling body, comprising:
providing a coupling body further comprising:
a reservoir internal to the coupling body,
a first surface including a first adhesive and a first plurality of openings extending between the reservoir and the first surface, and
a second surface including a second plurality of openings extending between the reservoir and the second surface;
adhering a first seal over the first surface;
adhering a second seal over the second surface; and
sealing the coupling body in a package;
wherein adhering the first seal over the first surface comprises adhering the first seal over the first plurality of openings and the adhesive on the first surface; and
wherein adhering the second seal over the second surface comprises adhering the second seal over the second plurality of openings on the second surface.

13. The method of claim 12, wherein the first surface is configured to adhere to an ultrasound patch device, the second surface includes a second adhesive, and wherein adhering the second seal comprises adhering the second seal over the second plurality of openings and the second adhesive on the second surface.

14. The method of claim 12, wherein the reservoir contains ultrasound gel and wherein the ultrasound gel includes a humectant.

15. The method of claim 12, wherein each of the first plurality of openings and the second plurality of openings is between approximately 100 microns and 500 microns in diameter and wherein the first plurality of openings and the second plurality of openings have a pitch of between approximately 1 mm and 5 mm.

16. The method of claim 12, wherein the first surface and the second surface of the coupling body comprise a same material.

17. The method of claim 12, wherein:
the coupling body includes an inner portion and an outer portion surrounding the inner portion, the inner portion including a hydrogel material;
the first surface and the second surface are on the outer portion of the coupling body; and
the reservoir is disposed between the inner portion and the outer portion.

18. The method of claim 17, wherein the outer portion comprises polypropylene.

19. The method of claim 12, wherein the first adhesive is coupled to a subpart of the first surface of the coupling body and wherein the subpart comprises a perimeter portion of the first surface of the coupling body.

20. The method of claim 12, wherein:
the coupling body is substantially rectangular;
the coupling body further comprises:
a middle portion,
a first end portion, and
a second end portion, and
wherein the first adhesive is coupled to at least one of the first end portion and the second end portion and not the middle portion; and
wherein the middle portion, the first end portion, and the second end portion have substantially a same length.
